SEQUENCE LISTING (14 sequences)

SEQ ID NO: 1 - DNA, 20 bp, Artificial Sequence
  Synthetic construct
agagtttgatcctggctcag

SEQ ID NO: 2 - DNA, 19 bp, Artificial Sequence
  Synthetic construct
ggttaccttgttacgactt

SEQ ID NO: 3 - DNA, 23 bp, Artificial Sequence
  Synthetic construct
taaaactyaaakgaattgacggg

SEQ ID NO: 4 - DNA, 23 bp, Artificial Sequence
  Synthetic construct
actgctgcsycccgtaggagtct

SEQ ID NO: 5 - DNA, 15 bp, Artificial Sequence
  Synthetic construct
yaacgagcgcaaccc

SEQ ID NO: 6 - DNA, 15 bp, Artificial Sequence
  Synthetic construct
gggttgcgctcgttg

SEQ ID NO: 7 - DNA, 21 bp, Artificial Sequence
  Synthetic construct
gactcctacgggaggcwgcag

SEQ ID NO: 8 - DNA, 20 bp, Artificial Sequence
  Synthetic construct
ccgtcaattcctttragttt

SEQ ID NO: 9 - DNA, 18 bp, Artificial Sequence
  Synthetic construct
ggattagataccctggta

SEQ ID NO: 10 - DNA, 20 bp, Artificial Sequence
  Synthetic construct
gactaccagggtatctaatc

SEQ ID NO: 11 - DNA, 19 bp, Artificial Sequence
  Synthetic construct
gtgccagcmgccgcggtaa

SEQ ID NO: 12 - DNA, 19 bp, Artificial Sequence
  Synthetic construct
gtattaccgcggctgctgg

SEQ ID NO: 13 - DNA, 19 bp, Artificial Sequence
  Synthetic construct
gagtttgatcmtggctcag

SEQ ID NO: 14 - DNA, 20 bp, Artificial Sequence
  Synthetic construct
cggttaccttgttacgactt

(19) United States
(12) Patent Application Publication
(10) Pub. No.: US 2019/0055542 A1
(43) Pub. Date: Feb. 21, 2019

(54) DETECTION OF MICROORGANISMS IN FLUIDS

(71) Applicant: OSP Microcheck Inc., Calgary (CA)

(72) Inventors: Stan Leong, Calgary (CA); Kim Dockens, Houston, TX (US); Shawna L. Johnston, Calgary (CA)

(21) Appl. No.: 16/079,240

(22) PCT Filed: Feb. 24, 2017
(86) PCT No.: PCT/CA2017/050241

§ 371 Date: Aug. 23, 2018

Related U.S. Application Data
(60) Provisional application No. 62/428,742, filed on Dec. 1, 2016.
(60) Provisional application No. 62/299,148, filed on Feb. 24, 2016.

(51) Int. Cl. C12N 15/10; C12Q 1/6888

(52) U.S. Cl. CPC C12N 15/1017; C12Q 1/6888

(57) ABSTRACT

Methods and kits for obtaining nucleic acid material from fluids comprising microorganisms are provided. The methods involve filtering the fluid sample through a filter, treating the filter with a lysis reagent to permit release of nucleic acid material from the microorganisms and adherence of the nucleic acid material to the filter, and elution of the nucleic acid material from the filter in an eluate. The methods are useful for monitoring and maintaining water quality, for example.

### CROSS-REFERENCE TO RELATED APPLICATION

This application claims the benefit of U.S. Provisional Patent Application No. 62/299,148 filed on Feb. 24, 2016 and U.S. Provisional Patent Application No. 62/428,742 filed on Dec. 1, 2016 and. The entire contents of U.S. Provisional Patent Application 62/299,148 and U.S. Provisional Patent Application No. 62/428,742 are hereby incorporated by reference in their entirety.

### FIELD OF THE DISCLOSURE

The present disclosure relates to methods for evaluating the quality of fluids. The present disclosure further relates to methods for the detection of microorganisms in water and other fluids, and more specifically to nucleic acid based methods of detecting microorganisms in water and other fluids.

### BACKGROUND OF THE DISCLOSURE

The following paragraphs are provided by way of background to the present disclosure. They are not however an admission that anything discussed therein is prior art or part of the knowledge of persons skilled in the art.

Throughout the world, there are many circumstances in which monitoring and maintaining fluid quality is important. For example, many pathogenic agents are water-borne, and in order to ensure a safe supply of drinking water for domestic purposes, such as drinking, food preparation and personal hygiene, the presence of such agents must be monitored and maintained within certain tolerances. Similarly, non-potable water, such as water used in the performance of industrial processes, must meet certain quality standards in order to be suitable for its intended use. The detection of microbial contaminants present in bodies of water in this regard represents a particular challenge.

It is well understood that techniques capable of assaying the microbial nucleic acid constituents present in fluids are highly desirable, since such techniques permit qualitative as well as quantitative identification of the microbial species and strains present therein. Upon identification, species-specific fluid treatment regimens may be designed and implemented in order to maintain or improve fluid quality.

Although several techniques for nucleic acid characterization present in water and other fluids have evolved, the heretofore known methodologies exhibit significant limitations. In many instances the shortcomings of known methodologies reflect the fact that the microbial constituency in a body of water is an inherently variable matrix. Thus when fluid conditions of a sample of which nucleic acid material is assayed deviate from those of the source fluid from which the fluid sample is drawn, the nucleic acid characterization no longer is representative of the in situ constituent microbial species or strains. Marked alterations in microbial constituency occur when fluid samples are obtained from locations where there is no access to near-by laboratory facilities, and storage and transport of the fluid sample is required. By way of example, when in a water sample the temperature fluctuates, a change in the constituent microbial species may ensue. By way of another example, in instances where water samples are obtained from anoxic environments, such as is the case when the quality of water present in subterranean water reservoirs is monitored, it is difficult to maintain such anoxic environment. The foregoing problems are further compounded by the fact that known methodologies for nucleic acid sample preparation are laborious and time-consuming. During the time required to prepare nucleic acid material using conventional methods, the microbial constituents are subject to change, leading to artifacts in nucleic acid characterization.

Furthermore, many known techniques aimed at characterization of nucleic acids present in fluids involve the isolation of nucleic acid material. This is frequently challenging since separation of non-microbial contaminants, for example, oil-based contaminants, minerals, suspended solids, or debris, from nucleic acid material is often difficult to achieve. The presence of contaminants subsequently interferes with the nucleic acid analysis.

In addition, there are other challenges associated with the use of water and other fluid samples as a source whence nucleic acid material is obtained. The disposal of fluid samples after nucleic acid analysis adds logistical complexity and associated costs. Fluid samples, including in some instances water samples, classified as dangerous goods are required to be disposed as outlined by applicable safety regulations, such as set forth in material safety data sheets. Furthermore shipping of fluid samples containing live microorganisms may be problematic for the receiving laboratory. Contamination of the fluid sample by the surrounding environment would impair the results of nucleic acid analysis conducted on the sample. Conversely, contamination of the surrounding environment by the live microorganisms from the fluid sample may be problematic depending on the sensitivity of the environment to the live microorganisms. Finally, it is noted that shipping of fluid samples is expensive and potentially dangerous, depending on the fluid sample and the mode of containing and shipping of the sample that is employed. Thus it will be clear from the foregoing that, even if a fluid sample can be secured for transport to an analytical laboratory, and a methodology to obtain representative nucleic acid material from such sample can be developed, many drawbacks associated with handling a fluid sample in the laboratory remain.

### SUMMARY OF THE DISCLOSURE

The following paragraphs are intended to introduce the reader to the more detailed description that follows and not to define or limit the claimed subject matter of the present disclosure.

In one aspect, the present disclosure relates to fluid quality, including water quality.

In another aspect, the present disclosure relates to methods for evaluating fluid quality by obtaining the nucleic acid constituents present in a fluid.

Accordingly, the present disclosure provides, in at least one embodiment, a method for obtaining nucleic acid material from a source fluid comprising microorganisms, the method comprising:
obtaining a fluid sample from a source fluid comprising microorganisms;
filtering the fluid sample through a filter having a pore size sufficiently small to prevent passage of at least some of the microorganisms through the filter to collect microorganisms associated with the filter;
treating the filter with a sufficient quantity of lysis reagent to release at least some of the nucleic acid material from the microorganisms associated with the filter and for the released nucleic acid material to adhere to the filter; and
treating the filter with a sufficient quantity of extractant to elute at least some of the nucleic acid material from the filter and to obtain an eluate comprising the nucleic acid material from the filter.

In another aspect, the present disclosure provides, in at least one embodiment, a method for evaluating the in situ quality of a fluid, the method comprising:
obtaining a fluid sample from a source fluid comprising microorganisms;
filtering the fluid sample through a filter having a pore size sufficiently small to prevent passage of at least some of the microorganisms through the filter to collect microorganisms associated with the filter;
treating the filter with a sufficient quantity of lysis reagent to release at least some of the nucleic acid material from the microorganisms associated with the filter and for the released nucleic acid material to adhere to the filter;
treating the filter with a sufficient quantity of extractant to elute at least some of the nucleic acid material from the filter and to obtain an eluate comprising the at least some of the nucleic acid material; and
analyzing the nucleic acid material in the eluate.

In some embodiments, the fluid sample is filtered under pressure.

In some embodiments, the fluid sample is obtained from a body of water and the method is a method for evaluating the in situ quality of the body of water.

In some embodiments, analyzing the nucleic acid material in the eluate is performed in a manner that permits the identification of a taxonomic order to which microorganisms present in the eluate belong, or the phylogenetic relationships between microorganisms.

In some embodiments, analyzing the nucleic acid material in the eluate comprises amplifying the nucleic acid material by PCR.

In some embodiments, analyzing the nucleic acid material in the eluate comprises obtaining nucleic acid sequence data.

In some embodiments, analyzing the nucleic acid material in the eluate comprises obtaining 16S rRNA nucleic acid sequence data.

In some embodiments, analyzing the nucleic acid material in the eluate comprises obtaining 16S rRNA nucleic acid sequence data and comparing the obtained sequence data with 16S rRNA nucleic acid sequence data present in a database comprising known 16S rRNA sequences.

In some embodiments, the data are recorded and stored on a computer readable medium.

In some embodiments, the method further involves a step of identifying and recording one or more classes of microorganisms present in situ in the source fluid.

In some embodiments, the classes of microorganisms are recorded and stored on a computer readable medium.

In another aspect, the present disclosure provides, in at least one embodiment, a method for obtaining nucleic acid material from a source fluid comprising microorganisms, the method comprising:
obtaining in a fluid transfer device a fluid sample from a source fluid comprising microorganisms;
coupling the fluid transfer device to a filter having a pore size sufficiently small to prevent passage of at least some of the microorganisms through the filter;
filtering the fluid sample through the filter thereby collecting the microorganisms on the filter;
decoupling the filter from the fluid transfer device a first time;
coupling a fluid transfer device comprising lysis reagent to the filter;
filtering the lysis reagent through the filter to release at least some of the nucleic acid material from the microorganisms on the filter and to adhere the released nucleic acid material to the filter; and
decoupling the filter from the fluid transfer device a second time to obtain the nucleic acid material adhered to the filter.

In a specific embodiment, the fluid transfer device is a syringe.

In another aspect, the present disclosure provides, in at least one embodiment, a kit for obtaining a nucleic acid sample, the kit comprising
a fluid transfer device to obtain a fluid sample from a source fluid comprising microorganisms;
a filter having a pore size sufficiently small to prevent passage of at least some of the microorganisms through the filter and to permit collection of the microorganisms associated with the filter; and
a lysis reagent which when filtered through the filter is capable of lysing microorganisms collected on the filter, releasing at least some of the nucleic acid material from the microorganisms, and adhering the released nucleic acid material to the filter.

In some embodiments, the fluid transfer device is a syringe.

In some embodiments, the kit further comprises an extractant for the elution of the nucleic acid material to obtain an eluate comprising nucleic acid material.

In yet another aspect, the present disclosure provides, in at least one embodiment, a use of a kit to evaluate the in situ identity of microorganisms in a fluid, the kit comprising:
a fluid transfer device to obtain a fluid sample from a source fluid comprising microorganisms;
a filter having a pore size sufficiently small to prevent passage of at least some of the microorganisms through the filter and to permit collection of the microorganisms associated with the filter; and
a lysis reagent which when filtered through the filter is capable of lysing microorganisms collected on the filter, release of at least some of the nucleic acid material from the microorganisms, and adherence of the released nucleic acid material to the filter.

Other features and advantages or the present disclosure will become apparent from the following detailed description. It should be understood, however, that the detailed description, while indicating preferred implementations of the present disclosure, is given by way of illustration only, since various changes and modification within the spirit and scope of the disclosure will become apparent to those of skill in the art from the detailed description.

### BRIEF DESCRIPTION OF THE DRAWINGS

For a better understanding of the various example embodiments described herein, and to show more clearly how these various embodiments may be carried into effect, reference will be made, by way of example, to the accompanying drawings which show at least one example embodiment, and the drawings will now be briefly described. It is noted that identical numbering of elements in different drawings is intended to refer to the same element, possibly shown situated differently, at a different size, or from a different angle. Thus, by way of example, element 28 in FIG. 1, FIG. 2A, FIG. 2B, FIG. 3A-FIG. 3C, FIG. 4A-FIG. 4C and FIG. 5A-FIG. 5C, refers in each instance to the same filter housing.

FIG. 1 shows a perspective view of a syringe and filter in accordance with one example embodiment of the present disclosure.

FIG. 2A-FIG. 2C show a schematic overview of certain steps performed in accordance with one embodiment of the present disclosure, notably steps involved in collection of a fluid sample using a syringe and coupling of a filter to the syringe.

FIG. 3A-FIG. 3C show a schematic overview of certain steps performed in accordance with one embodiment of the present disclosure, notably steps involved in filtering of a fluid sample through a syringe.

FIG. 4A-FIG. 4C show a schematic overview of certain steps performed in accordance with one embodiment of the present disclosure, notably steps involved in treating of a filter with lysis reagent.

FIG. 5A-FIG. 5C show a schematic overview of certain steps performed in accordance with one embodiment of the present disclosure, notably steps involved in treating of a filter with extractant.

FIG. 6 shows a bar graph representing the results of a 16s rRNA sequence analysis of various fluid fractions (each represented by a bar) obtained from a first water sample. Each grey shaded box within a bar represents a different microbial genus and the size of each box is proportional to the relative quantity of a microbial genus present in a fraction. Identically shaded boxes signify the presence of identical genera in samples. The graphed line represents a plot of the PCR yield of 16s amplifications in ng/μL as shown on the right axis. Specific yield values for each sample are noted.

FIG. 7 shows a graph representing the results of a principal component analysis of various fluid fractions obtained from a first water sample.

FIG. 8 shows a bar graph representing the results of a 16s rRNA sequence analysis of various fluid fractions (each represented by a bar) obtained from a second water sample. Each grey shaded box within a bar represents a different microbial genus and the size of each box is proportional to the relative quantity of a microbial genus present in a fraction. Identically shaded boxes signify the presence of identical genera in samples. The graphed line represents a plot of the PCR yield of 16s amplifications in ng/μL as shown on the right axis. Specific yield values for each sample are noted.

FIG. 9 shows a graph representing the results of a principal component analysis of various fluid fractions obtained from a second water sample.

FIG. 10 shows results obtained following extraction of nucleic acid material from various samples prepared as described in Example 3, using water samples obtained from various sources as indicated in Table 5.

FIG. 11A-FIG. 11B show bar graphs representing the results of a 16s rRNA sequence analysis of various samples (each represented by a bar). Each grey shaded box within a bar represents a different microbial genus and the size of each box is proportional to the relative quantity of a microbial genus present in a fraction. Identically shaded boxes signify the presence of identical genera in samples. FIG. 11A represents results from a water sample obtained from Source ID F (see: Table 5). FIG. 11B represents results from a water sample obtained from Source ID H (see: Table 5)

FIG. 12A-FIG. 12B shows a graph representing the results of a principal component analysis of various fluid fractions obtained from a first water sample. FIG. 12A represents results from a water sample obtained from Source ID F (see: Table 5). FIG. 12B represents results from a water sample obtained from Source ID H (see: Table 5)

FIG. 13 shows results derived from 16S metagenomics data (genus level) compiled as a heat map to allow for visual comparison of sample microbial community similarity obtained from sources described in Table 5. The darkest grey fields denote the highest level of similarity to the original microbial community (>5 of 8 matched), while the lightest fields denote the least (<5 matched to original community) level of similarity.

The drawings together with the following detailed description make apparent to those skilled in the art how the disclosure may be implemented in practice.

### DETAILED DESCRIPTION OF THE DISCLOSURE

Various processes, methods and systems will be described below to provide an example of an embodiment of the claimed subject matter. No embodiment described below limits any claimed subject matter and any claimed subject matter may cover processes, methods, or systems that differ from those described below. The claimed subject matter is not limited to any process, method, or system having all of the features of processes, methods, or systems described below, or to features common to multiple or processes, methods, compositions or systems described below. It is possible that a process, method, or system described below is not an embodiment of any claimed subject matter. Any subject matter disclosed in processes, methods, or systems described below that is not claimed in this document may be the subject matter of another protective instrument, for example, a continuing patent application, and the applicants, inventors or owners do not intend to abandon, disclaim or dedicate to the public any such subject matter by its disclosure in this document.

All publications, patents, and patent applications referred herein are herein incorporated by reference in their entirety to the same extent as if each individual publication, patent or patent application was specifically indicated to be incorporated by reference in its entirety.

### Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“Aqueous fluid”, as used herein, can refer to a fluid whose primary composition is water-based, including but not limited to fluids comprising at least approximately 95%, 96%, 97%, 98%, 99%, 99.5%, 99.6%, 99.7%, 99.8% or 99.9% water.

“Body of water”, as used herein, can refer to any quantity of water and includes any natural or artificial quantity of water including by way of example, and not limitation, water present in any ocean, sea, bay, river, stream, creek, lake, pond, canal, ditch, reservoir, basin, pool, channel, conduit, vessel, apparatus, system or receptacle. The term further refers to potable and non-potable bodies of water.

“Microorganism”, as used herein, includes but is not limited to, all bacterial cells, archaeabacterial cells, fungal cells, yeast cells, and algal cells, and microorganisms may be unicellular or multicellular in nature.

“Class(es) of microorganisms”, as used herein, can refer to divisions in groups of microorganisms in accordance with any kind of classification scheme including, but not limited to, taxonomic order (i.e. kingdom, phylum, class, order, family, genus, species or strain), metabolism, for example, sulfate reduction, nitrogen fixation etc., pathogenicity, morphology, for example, cell shape or Gram-staining, or groups of microorganisms based any other classification scheme.

“Lysis reagent”, as used herein, can refer to a reagent capable of dissolving the integrity of cell membranes and/or cell walls of microorganisms.

It should be noted that terms of degree such as, “essentially” “about”, “near” and “approximately”, as used herein, mean a reasonable amount of deviation of the modified term such that the end result is not significantly changed. These terms of degree should be construed as including a deviation of the modified term if this deviation would not negate the meaning of the term it modifies.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

### General Implementation

As hereinbefore mentioned, the present disclosure provides methods for obtaining samples of nucleic acid material from fluids. By providing methods of obtaining representative samples of nucleic acid material, the methods can be of great value in overcoming barriers associated with known methods. The present disclosure allows the isolation of a sample of nucleic acid material of which the composition can surprisingly accurately reflect the composition of the nucleic acid constituents present in the larger body amount of fluid, such as a body of water, from which the sample is drawn. Thus when the nucleic acid material is subsequently used for qualitative and/or quantitative characterization, the results obtained accurately correspond with the nucleic acid material present in the source fluid from which the sample is drawn, and permits an accurate qualitative and/or quantitative assessment of microorganisms present in situ. The herein disclosed methods can be rapid and easy to perform, and may be conducted, at least in part, in close proximity of the source fluid from which the fluid sample is obtained. Thus the methods of the present disclosure are particularly useful in settings where no laboratory or laboratory equipment is available for immediate analysis. In addition, the methods of the present disclosure do not necessarily require the transportation of water, and thus these methods can be inexpensive and safe to conduct.

There is a need in the art a need for rapid, easy to perform, and accurate methods to identify the constituent microorganisms present in fluids.

Accordingly, the present disclosure provides, in at least one embodiment, a method for obtaining nucleic acid material from a source fluid comprising microorganisms, the method comprising:
obtaining a fluid sample from a source fluid comprising microorganisms;
filtering the fluid sample through a filter having a pore size sufficiently small to prevent passage of at least some of the microorganisms through the filter to collect microorganisms associated with the filter;
treating the filter with a sufficient quantity of lysis reagent to release at least some of the nucleic acid material from the microorganisms associated with the filter and for the released nucleic acid material to adhere to the filter; and
treating the filter with a sufficient quantity of extractant to elute at least some of the nucleic acid material from the filter and to obtain an eluate comprising the nucleic acid material from the filter.

Accordingly, the present disclosure, in one aspect, involves providing a fluid sample obtained from a source fluid comprising microorganisms. In accordance herewith, any natural or artificial source of the source fluid may be used. In certain embodiments, the fluid sample is a water sample obtained from a large body water, including by way of example, but not limitation, a naturally occurring large body of water, such as an ocean, sea, bay, lake, river, stream, creek or channel, natural subterranean reservoir, or a man-made large body of water such as a pond, pool, reservoir, man-made subterranean reservoir, canal, or ditch. The body of water may be deemed potable, for example, water obtained from a municipal water drinking water system, or non-potable, for example, water obtained from a municipal sewage system, or industrial wastewater system. In other embodiments, the fluid sample is obtained from a less voluminous quantity of the source fluid, for example, a fluid present in a vessel, container, tank, conduit or receptacle used in an industrial, laboratory or domestic environment. In some embodiments, the fluid sample is an aqueous fluid sample, however in other embodiments, the fluid sample is non-aqueous, for example a fluid obtained from an industrial site where non aqueous fluids are employed, for example a petroleum product recovery site or a refinement site.

In order to obtain a sample of the fluid, any suitable fluid sampling technique and fluid sampling devices known to the art may be used. Thus, for example, a container, vial, syringe, tube or receptacle capable of drawing fluid from the source fluid and containing an aliquot of the source fluid may be used, and such devices may generally be referred herein as “fluid transfer devices”. In general terms, the sampling device is contacted with the source fluid to draw and transfer an aliquot of the source fluid to the sampling device, and then the sampling device containing the fluid aliquot is separated from the source fluid to obtain a fluid sample. In certain embodiments, the sampling technique and device provide a fluid sample which is substantially free of larger particulate matter and debris, for example, substantially free of particulate matter and debris larger in size than 100 μm, more preferably larger in size than about 10 μm. The skilled artisan will readily recognize that the volume of the sample of fluid may vary, depending, for example, in part on the volume of the source fluid, and that fluid sampling techniques may vary depending on the source fluid, and may be adjusted as desirable.

The source fluid, in accordance herewith, is further characterized in that it comprises microorganisms. In some embodiments, the microorganisms may be of a single taxonomic order, for example a single kingdom, phylum, class, order, family, genus, species or strain. In other embodiments, the microorganisms may represent a plurality of kingdoms, phyla, classes, orders, families, genera, species or strains. Thus the phylogenetic relationship between the microorganisms may vary. The kingdom, phylum, class, order, family, genus, species or strains of microorganisms may vary, and will, as will readily be appreciated by those of skill in the art, depend on the source of the fluid. The microorganisms may be pathogenic or represent other health or safety risks to humans or animals, or the microorganisms may cause operational challenges in the performance of industrial processes. In other embodiments, the microorganisms are desirable, for example, certain desirable microorganisms performing certain catabolic or anabolic processes, for example, the degradation of waste products, including, for example, in wastewater treatment facilities.

The methods of the present disclosure, in one aspect, involve filtering a fluid sample through a filter. In accordance herewith, the filter is selected to have a pore size sufficiently small to prevent passage of at least some of the microorganisms through the filter, and sufficiently large for all or substantially all of the other constituents of the fluid sample to substantially pass through the filter. In preferred embodiments, the filter is selected to have a pore size sufficiently small to prevent passage of most, substantially all, or all of the microorganisms through the filter, and sufficiently large for all or substantially all of the other constituents of the fluid sample to substantially pass through the filter. In some embodiments, the filter is selected to have a pore size sufficiently small to prevent passage of at least about 90%, 95%, 96%, 97%, 98%, 99%, 99.5%, 99.9%, 99.99% of the microorganisms present in the filtered fluid. Pore sizes in accordance herewith can be smaller than about 100 μm, more preferably smaller than about 10 μm, and most preferably in the range from about 10 μm to about 0.1 μm, for example, about 10 μm, 9 μm, 8 μm, 7 μm, 6 μm, 5 μm, 4, μm, 3 μm, 2 μm, 1 μm, 0.9 μm, 0.8 μm, 0.7 μm, 0.6 μm, 0.5 μm, 0.4 μm, 0.3 μm, 0.2 μm or about 0.1 μm. At such pore sizes the fluid sample of the present disclosure, including its non-microbial constituents, such as minerals, suspended solids, organic materials, and oils, can substantially pass through the filter, while microorganisms will be retained and collected on the filter.

In general terms, the filter in accordance with the present disclosure comprises a porous portion, a fluid inlet portion and a fluid outlet portion. In order to filter the fluid sample, a portion or substantially all of the fluid sample, for example, a volume in the range from about 0.5 ml to about 100 ml, is transferred from the fluid transfer device and applied to the fluid inlet portion of the filter, and then permitted to gradually pass through the porous portion of the filter and exit the filter via the fluid outlet portion. In some embodiments, gravitational force alone facilitates fluid flow through the filter. In other embodiments, an exogenous force is exerted on the fluid to effect fluid flow through the filter, and the fluid is filtered under pressure. As used herein the term “filtered under pressure” is intended to mean that an exogenous pressure is applied to the fluid to effect fluid passage through the filter. Such exogenous pressure may be applied to the fluid using any device for applying pressure to the fluid, referred to hereinafter as a “pressurizing device”. Filtering the fluid under pressure may be advantageous since under such conditions the integrity of cellular membranes of the microorganisms present in the sample fluid may be weakened. In accordance herewith the pressurizing device is preferably a manually operated pressurizing device, such as, for example, a syringe, as hereinafter further detailed. Generally only fluid constituents larger in size than the pore size of the porous portion of the filter are retained. Most of the fluid will pass through the filter and exit via the fluid outlet portion, after which the filtered fluid optionally may be collected. In typical examples, at least 90%, 95%, 97%, 98%, or 99% of the sample fluid is passed through the filter.

In accordance herewith, the filter of the present disclosure can be further selected to permit adherence of nucleic acid material thereto, notably to the surface area of the filter. In some embodiments, the filter comprises a material chosen from: a polymer, such as, but not limited to, for example, a polyester, a polyether, a cellulose, a polyethersulfone, a nylon, a polyacrylate, a polyalkylacrylate, a polyalkylene, a polyimide, a polycarbonate, a polyphenylene, a polynaphthalene, a polysilsesquioxance, a polysiloxane, a polysaccharide, derivatives thereof, porous variants thereof, and substituted variants thereof e.g., halogenated and alkylated variants thereof, and the like, ionomers thereof, and copolymers thereof, or a glass e.g., a silicate, a borosilicate, an aluminate, a zeolite, and porous variants thereof.

In one example embodiment, the filter is provided in the form of a silica (SiO₂) fiber based matrix material, including, for example, in preferred specific embodiments, in the form of a silicate or borosilicate glass microfiber filter. Such filters have been found to be particularly suitable, in that, upon treatment of these filters with the lysis reagent, nucleic acid material released from microorganisms readily adheres thereto.

Alternatively, in some embodiments, suitable filters that further may be used in accordance herewith include filters comprising cellulose acetate (CA) material, a polytetrafluorethylene (PTFE) material, a nylon material, or a polypropylene material.

The filters of the present disclosure may be provided in any suitable geometric dimension, for example, in one embodiment, as a column or, in another embodiment, in the form of a membrane, for example, a circular membrane having a thickness between about 100 μm and 750 μm. All geometric forms and shapes of filters are encompassed by the present disclosure.

In some embodiments, multiple silica microfiber membranes are stacked to form a stacked silica microfiber glass filter, for example, a stacked silicate or borosilicate glass microfiber membrane filter.

The filter and filtering conditions can be optimized or adjusted, for example by preparing a plurality of fluid samples and a plurality of filters and/or filtering conditions, and recovering eluate as herein described, and evaluating the amount of nucleic acid material obtained in the eluate. Then, a filter and filtering conditions can be selected that provide the most desirable effect, including, for example, a filter and filtering conditions which result in adherence of all or substantially all of the nucleic acid material present in the fluid sample to the filter, and for all or substantially all of the other constituents of the fluid sample to substantially pass through the filter.

In accordance herewith, in one aspect, the filter is treated with a sufficient quantity of lysis reagent to release at least some of the nucleic acid material from the microorganisms and for the released nucleic acid material to adhere to the filter. In preferred embodiments, the filter is treated with a sufficient quantity of lysis reagent to release most, substantially all, or all of the nucleic acid material from the microorganisms and for the released nucleic acid material to adhere to the filter. In some embodiments, the filter is treated with a sufficient quantity of lysis reagent to release at least about 90%, 95%, 96%, 97%, 98%, 99%, 99.5%, 99.9%, 99.99% of the total nucleic acid material from the microorganisms associated with the filter. Upon treatment with lysis reagent, the microorganisms associated with the filter generally lose viability and are no longer able to grow or multiply. The lysis reagent used in accordance with the present disclosure can vary and in some embodiments are detergents, cell-wall lysis enzymes, reducing reagents and mixtures or combinations thereof. The lysis reagents are preferably included in an amount effective for cell lysis, without inhibiting adherence of released nucleic acid material to the filter, or flow through of non-nucleic acid material.

In embodiments hereof wherein the lysis reagent is a detergent, the detergent can be nonionic, ionic, or zwitterionic, but nonionic and zwitterionic detergents are preferred. Example detergents include Triton X-100; Triton X-114; NP-40; Brij-35; Brij-58; Tween 20; Tween 80; octyl glucoside; octyl thioglucoside; sodium dodecyl sulfate (SDS); 3-[(3-cholamidopropyl)dimethylammonio]-l-propanesulfonate (CHAPS); 3-[(3-cholamidopropyl)dimethylammonio]-2-hydroxy-l-propanesulfonate (CHAPSO); alkyldimethylbenzylammonium chloride (ADBAC); and didecyldimethylbenzylammonium chloride (DDAC).

In embodiments hereof wherein the lysis reagent is a cell-wall lysis enzyme, the cell wall lysis enzyme can be a lyase, e.g. peptidoglycan lyase; a muramidase, e.g. β-N-acetylmuramidase; or a glucanase, e.g. β-l, 3-glucanase.

In embodiments hereof wherein the lysis reagent is a reducing reagent, the reducing reagent can be dithioerythreitol (DTE), β-mercaptoethanol, cysteamine, sodium sulphite, and tris(2-carboxyethyl)phosphine (TCEP) and dithiothreitol (DTT).

In one particular embodiment, the lysis reagent is an aqueous solution comprising a mixture of trisodium phosphate dodecahydrate, 1-5%; sodium hydroxide, 0.1-1%; and alkyl dimethyl benzyl ammonium chloride, 0.1-1%.

Treatment of the filter may be achieved by passing lysis reagent through the filter, or in other embodiments, by immersing and incubating the filter in lysis reagent. In some embodiments, the filter can be incubated with the lysis reagent at elevated temperatures e.g. for 5 to 15 minutes at 95° C. The amount of lysis reagent, the composition of the lysis reagent and the lysis conditions can be optimized or adjusted, for example, by preparing a plurality of fluid samples and a plurality of lysis reagents, filtering and lysing the microorganisms using the different lysis reagents and/or different lysis conditions, and evaluating the amount of nucleic acid material obtained following extraction. Then, a lysis reagent and lysis conditions can be selected that provide the most desirable effect, including, for example, a lysis reagent and lysis conditions which result in adherence of all or substantially all of the nucleic acid material present in the fluid sample to the filter, and/or a lysis reagent and lysis conditions which in the performance of subsequent steps provide an eluate comprising all or substantially all of the microbial nucleic acid material present in the fluid sample and/or a lysis reagent and lysis conditions which result in the subsequent recovery of an eluate comprising nucleic acid material substantially free of other cellular constituents.

Nucleic acid material in the present disclosure includes, any nucleic acid material encompassed by microorganisms and includes polynucleotides and oligonucleotides, including DNA and/or RNA, mRNA, rRNA; genomic DNA; plasmids; genes and gene fragments.

In accordance herewith, in one aspect, the filter is treated with an extractant to elute at least some of the nucleic acid material from the filter. Preferably most, substantially all, or all, of the nucleic acid material is eluted from the filter to obtain an eluate comprising the nucleic acid material. The extractant, used in accordance with the present disclosure, may vary and includes, but is not limited to, water-based extractants such as for example distilled water, molecular grade water, Tris-EDTA (TE) (10 mM Tris-HCl, 1.0 mM EDTA, pH 8-9), Tris-HCl buffer (e.g. 10 mM, pH 8-9) or mixtures or combinations thereof. The amounts used are amounts effective at extracting and eluting preferably, most, substantially all or all of the nucleic acid material from the filter. Extraction may be accomplished by passing extractant through the filter or, in other embodiments, by immersing and incubating the filter in extractant, and eluting the nucleic acid material from the filter and thus obtain an eluate comprising nucleic acid material.

In some embodiments, treatment with extractant can be repeated two or more times in order to ensure elution of all or substantially all of the nucleic acid material. The amount of extractant, the composition of the extractant, and extraction conditions can be optimized or adjusted, for example by preparing a plurality of fluid samples and a plurality of extractants, filtering and lysing the microorganisms using the different extractants, and evaluating the amount of nucleic acid material obtained following extraction. Then, an extractant may be selected that provides the most desirable effect, including, for example, an extractant and extraction conditions which result in release of all or substantially all of the nucleic acid material present in the fluid sample from the filter, and/or an extractant and extraction conditions which result in the subsequent recovery of an eluate comprising nucleic acid material substantially free of other cellular constituents.

In some embodiments, the eluate is treated to remove contaminating non nucleic acid constituents remaining in the eluate to thereby obtain a purified nucleic acid preparation, for example, a nucleic acid preparation containing no more than approximately 5%, 4%, 3%, 2%, 1%, 0.5%, or 0.1% (w/w) of non-nucleic acid constituents. Thus protein removal techniques, for example using precipitation agents, such as a flocculant (e.g. a salt, such as, ammonium acetate or magnesium chloride); filtering techniques, for example column filtration (using e.g. a polyvinylpolypyrrolidone spin column, or size-exclusion chromatography using e.g. a SEPHADEX G-200™ or MICROSPIN S-400 HR™); nucleic acid capture on magnetic beads; and nucleic acid precipitation techniques (e.g. ethanol precipitation), or a combination of the foregoing can be used to obtain a further purified nucleic acid preparation. Methods and techniques for the treatment of an eluate sample to remove non-nucleic constituents that can be used in accordance herewith include those described in U.S. Pat. No. 7,459,548.

The eluate obtained in accordance with the present disclosure comprises preferably at least about 90%, 95%, 96%, 97%, 98%, 99%, 99.5%, 99.9%, 99.99% (w/w) of the total nucleic acid material of the microorganisms initially captured on the filter. In some embodiments, the nucleic acid material obtained in the eluate or purified nucleic acid preparation, as the case may be, is substantially free of other cellular constituents and other contaminants. Thus in preferred embodiments, the eluate or purified nucleic acid preparation, contains no more than approximately 5%, 4%, 3%, 2%, 1% (w/w) of non-nucleic acid constituents. The extent to which the eluate or purified nucleic acid preparation is substantially free of non-nucleic acid constituents can be evaluated in various ways, including for example spectrophotometrically by determining ultraviolet light absorption at 260 nm and 280 nm, and the ratio of absorbance at these two wavelengths (A_{260/280}). In preferred embodiments, the eluate exhibits an A_{260/280} within the range from about 1.7 to about 2.2. Furthermore, the nucleic acid material obtained in accordance with the performance of the methods of the present disclosure, is representative of the nucleic acid constituents present in situ in the source fluid.

In some embodiments, prior to treatment of the filter with lysis reagent, the filter is washed using a washing reagent in order to remove fluid contaminants, for example, minerals, salts, oil or grease contaminants, suspended solids, or debris. Suitable washing reagents that can be used in the regard include for example, a chloride salt based washing reagent, such as a sodium chloride; a guanidium chloride based washing reagent; dimethyl sulfoxide (DMSO) (60-100% v/v); acetone (e.g. 90% v/v) or ethanol, or a combination of the foregoing.

In some embodiments, following treatment of the filter with lysis reagent, and prior to treatment of the filter with extractant, the filter is washed once or several times using a suitable washing reagent, including any of the herein before described washing reagents. Treatment of the filter with washing reagent may result in further removal of cellular debris, i.e. non-nucleic acid cellular constituents from the filter, and thus result in an eluate comprising smaller quantities of non-nucleic acid cellular constituents.

As hereinbefore noted, the present disclosure relates, in another aspect, to fluid quality and methods of evaluating fluid quality, and in particular to methods of evaluating microorganisms in fluids. Accordingly, the present disclosure further provides a method for evaluating the in situ quality of a fluid, the method comprising:
obtaining a fluid sample from a source fluid comprising microorganisms;
filtering the fluid sample through a filter having a pore size sufficiently small to prevent passage of at least some of the microorganisms associated with the filter through the filter to collect microorganisms associated with the filter;
treating the filter with a sufficient quantity of lysis reagent to release at least some of the nucleic acid material from the microorganisms and for the released nucleic acid material to adhere to the filter;
treating the filter with a sufficient quantity of extractant to elute at least some of the nucleic acid material from the filter and to obtain an eluate comprising the at least some of the nucleic acid material; and
analyzing the nucleic acid material in the eluate.

In accordance with the foregoing, the nucleic acid material in the eluate is analyzed to thereby assess the in situ quality of the source fluid from which the fluid sample was obtained. A wide range of qualitative and quantitative nucleic acid analysis methodologies are encompassed in the present disclosure. In preferred embodiments, nucleic acid material is analyzed in a manner that permits the identification of classes of microorganisms present in the eluate, for example, by identification of a taxonomic rank to which the microorganisms present in the eluate belong. Methods of analysis include, but are not limited to, methods permitting the identification of microorganisms belonging to different taxonomic ranks, including, for example, different kingdoms, phyla, classes, orders, families, genera, species or strains of microorganisms, or the determination of phylogenetic relationships between microorganisms, which can be visualized in the form of a phylogenetic tree. Such identification can be based on a variety of techniques, including e.g. polynucleotide primer based nucleic acid analysis techniques, such as polymerase chain reaction (PCR) based techniques, including quantitative PCR (qPCR) based amplification techniques, isothermal amplification based techniques, as well as methods that permit the determination of nucleic acid sequence identity, such as primer based nucleic acid sequence methodologies, and ribosomal RNA (rRNA) based nucleic acid identification techniques are used.

In some embodiments, the nucleic acid material in the eluate is amplified through, for example, a PCR reaction and using one or more pairs of PCR primers capable of amplifying a target sequence, in sufficient amounts for the amplification of target polynucleotides, if present. PCR methods are well known to those of skill in the art, for example as described in: PCR Troubleshooting and Optimization: The Essential Guide, Kennedy and Oswald (ed.s), Caister Academic Press (2011), and PCR Basics, McPherson and Moller, Taylor and Francis (New York) (2006). PCR methodology, reagents, and devices can vary substantially, and it is within the skill of one in the art to determine the proper conditions for an effective amplification reaction, for example, the sufficient concentration of a given primer pair, enzyme selections and amounts, temperature conditions, the number of cycles required, and the proper preparation of bacterial DNA samples.

In some embodiments, the nucleic acid material in the eluate is amplified using one or more primers or primer pairs capable of amplifying 16S rRNA sequences, to obtain amplified 16S rRNA material. In some embodiments, any one of the primers set forth in SEQ ID NO: 1 to SEQ ID NO: 14 is used for such amplification.

In some embodiments, the nucleic acid material is amplified using primers capable of identifying a class of microorganisms, for example, a specific taxonomic order of microorganisms, such as a kingdom, phylum, class, orders, family, genus, species or strain. Thus in some embodiments, primers directed to a specific pathogenic species of microorganism can be used and the nucleic acid material can be evaluated for the presence of such specific pathogenic species. From the presence or absence of an amplified sequence obtained following amplification of the nucleic acid material in the eluate, the presence or absence, respectively, of a taxonomic order of microorganism, i.e. a kingdom, phylum, class, orders, family, genus, species or strain, in situ in the source fluid can be inferred.

In some embodiments, the nucleic acid material is amplified using primers capable of identifying classes of microorganisms capable of performing a specific metabolic function, for example, the formation of methane, the reduction of sulfate, the fixation of nitrogen etc. Microorganisms thus identified, for example sulfate reducing bacteria, methanogens, or nitrogen fixing bacteria may belong to the same taxonomic order or to different taxonomic orders, however they can be inferred to share the capability to perform a specific metabolic function. From the presence or absence of an amplified sequence obtained following amplification of the nucleic acid material in the eluate, the presence or absence, respectively, of microorganisms capable of performing a specific metabolic function in situ in the source fluid can be inferred.

In some embodiments, the nucleic acid material in the eluate is used to obtain nucleic acid sequence data.

In some embodiments, the nucleic acid material in the eluate is used to obtain 16S rRNA sequence data using 16S rRNA based nucleic acid sequence identification techniques. Polynucleotide primer sequences, and one or more pairs of sequences, that can be used for amplification of 16S rRNA sequences present in the nucleic acid material in the eluate include the sequences comprising or consisting of SEQ ID NO: 1 to SEQ ID NO: 14 set forth herein. The obtained amplified polynucleotides can be sequenced using nucleic acid sequencing techniques, with which the skilled artisan will be familiar, and the obtained sequences can used to identify similar or identical sequences in a 16S rRNA database comprising 16S rRNA sequences of classified microbial species, for example, EzTaxon-e (http://eztaxon-e.ezbiocloud.net), the Ribosomal Database Project (http://rdp.cme.msu.edu/), or Mothur (http://www.mothur.org; Schloss P. D. et al., Applied and Environmental Microbiology 75 (23) 7537-7541), to thereby identify the class of microorganism to which microorganisms present in the sample belong, and assess the in situ quality of the fluid from which the sample was obtained.

Further techniques for nucleic analysis that can be used in accordance with the present disclosure are described in Molecular Cloning, Green and Sambrook, 2012, Cold Spring Harbor Laboratory Press.

As hereinbefore noted, the methodologies of the present disclosure can be performed in close proximity of the source fluid from which the fluid sample is obtained. Thus it is possible to perform the methodologies of the present disclosure immediately upon obtaining a fluid sample from a source fluid.

In some embodiments, the method of the present disclosure up to and including the step comprising:
treating the filter with a sufficient quantity of lysis reagent to release at least some of the nucleic acid material from the microorganisms associated with the filter and for the released nucleic acid material to adhere to the filter;

is performed within less than about 5 minutes, about 10 minutes, about 30 minutes, about 1 hour or about 2 hours from obtaining the fluid sample.

In some embodiments, the method of the present disclosure up to and including the step comprising
treating the filter with a sufficient quantity of lysis reagent to release at least some of the nucleic acid material from the microorganisms associated with the filter and for the released nucleic acid material to adhere to the filter;

is performed within less than about 5 minutes, about 10 minutes, about 30 minutes, about 1 hour or about 2 hours from obtaining the fluid sample, and thereafter the filter is stored for at least 1, 2, 3, 4, 5, 6, 7, 8, 9, 10, 11, 12, 13 or 14 days, prior to performing the step comprising:
treating the filter with a sufficient quantity of extractant to elute at least some of the nucleic acid material from the filter and to obtain an eluate comprising the at least some of the nucleic acid material.
   The filter can be stored at room temperature, or more preferably, at a lower temperature, for example, at about 4° C.

In some embodiments, the method of the present disclosure up to and including the step comprising:
treating the filter with a sufficient quantity of extractant to elute at least some of the nucleic acid material from the filter and to obtain an eluate comprising the at least some of the nucleic acid material;

is performed within less than about 5 minutes, about 10 minutes, about 30 minutes, about 1 hour or about 2 hours from obtaining the fluid sample.

As hereinbefore noted, the nucleic acid material obtained in accordance with the performance of the methods of the present disclosure can be representative of the nucleic acid constituents present in situ in the source fluid. With the term “representative of the nucleic acid constituents present in situ in the source fluid”, it is meant that the nucleic acid constituents present in the sample fluid are qualitatively and quantitatively approximately similar to the nucleic acid constituents in situ in the source fluid, at the time the sample fluid was obtained. Thus, by way of example only, if in a sample fluid an amount y/milliliter of nucleic acid constituents belonging to bacterial species “a” and z/milliliter of nucleic acid constituents belonging to bacterial species “b” are identified, the source fluid in situ will equally contain approximately y/milliliter of nucleic acid constituents belonging to bacterial species “a” and z/milliliter of nucleic acid constituents belonging to bacterial species “b”. In certain embodiments, concentrations of nucleic acid constituents can be converted to concentrations of bacterial species, and expressed in for example number of bacteria/ml of fluid.

In some embodiments, the methods further involve a step of identifying one or more classes of microorganisms present in situ in the source fluid, and providing information (i.e. data) related thereto, such as, but not limited to, for example, data relating to the absolute or relative quantities of microorganisms belonging to one or more classes of microorganisms present in situ in the source fluid, data relating to metabolic processes performed by the identified microorganisms, data relating to risks associated with the identified microorganisms, such as, for example, data relating to machine or facility operational risks, human or animal health risks, or data relating to human or animal diseases.

Thus it will be clear from the foregoing that the performance of the methods of the present disclosure can provide data with respect to the presence of microorganisms in the fluid sample, as well as in situ in a source fluid, including the classes to which microorganisms present in the fluid sample and in situ in the source fluid belong, and other herein mentioned data related thereto. In some embodiments, the obtained data can be recorded and stored. In some embodiments, the obtained data is received as input by a computer system, such as, for example, a desktop computer, a laptop, a tablet, a cellular phone, a smart phone, and recorded and stored on the memory of a computer system. The computer system further comprises a processor encoding one or more programs causing processing of the data, including, for example, programs to organize or analyze the data, programs to display the data on a computer screen, for example in the form of tables, graphs, text or figures, programs for a user to manipulate the data, programs to transmit the data over a network, or programs to print the data. The data obtained in accordance with the performance of methods of the present disclosure can further be recorded and stored on a computer readable medium, such as a disk (floppy diskette or hard disk), optical CD such as CD- or DVD-ROM/RAM, magnetic tape, electrical storage media such as RAM and ROM, and hybrids of these such as magnetic/optical storage media. In other embodiments, the obtained data can be recorded and stored in the form of printed data, for example, in the form of data written or printed on paper.

In accordance with the foregoing, in one aspect, the present disclosure further provides, in one embodiment, a computer readable medium comprising data relating to microorganisms present in situ in a source fluid wherein the data has been obtained by analysis of nucleic acid material in an eluate obtained in accordance with the methods of the present disclosure. The data can include, without limitation, nucleic acid sequence data, including 16S rRNA nucleic acid sequence data, classes of microorganisms, such as the taxonomic order, e.g. the kingdom, phylum, class, order, family, genus, species or strain, the relative or absolute quantity of a microorganism, or phylogenetic relationships between two or more microorganisms.

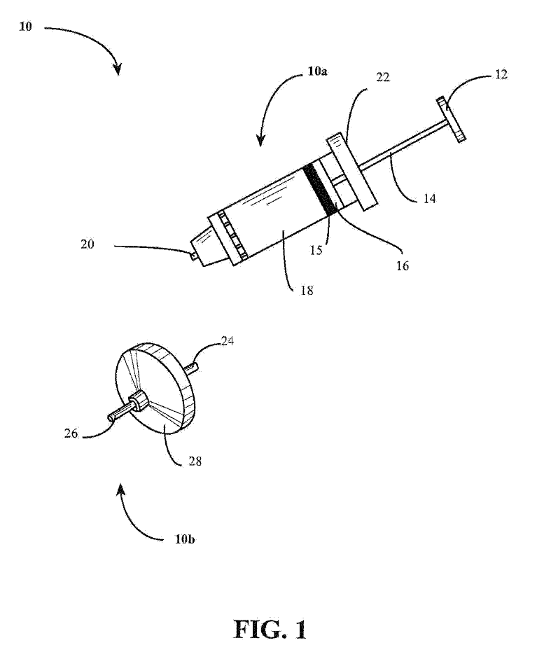

The present disclosure provides, in at least one specific example embodiment, a method involving the use of a fluid transfer device to obtain a fluid sample, together with a filter which can be reversibly coupled to the fluid transfer device. In specific examples of such embodiments, the fluid transfer device is a syringe, for example, a syringe having a fluid capacity anywhere in the range of from 1 ml to 250 ml. The foregoing embodiment of the present disclosure is hereinafter further illustrated with reference to FIG. 1 to FIG. 5.

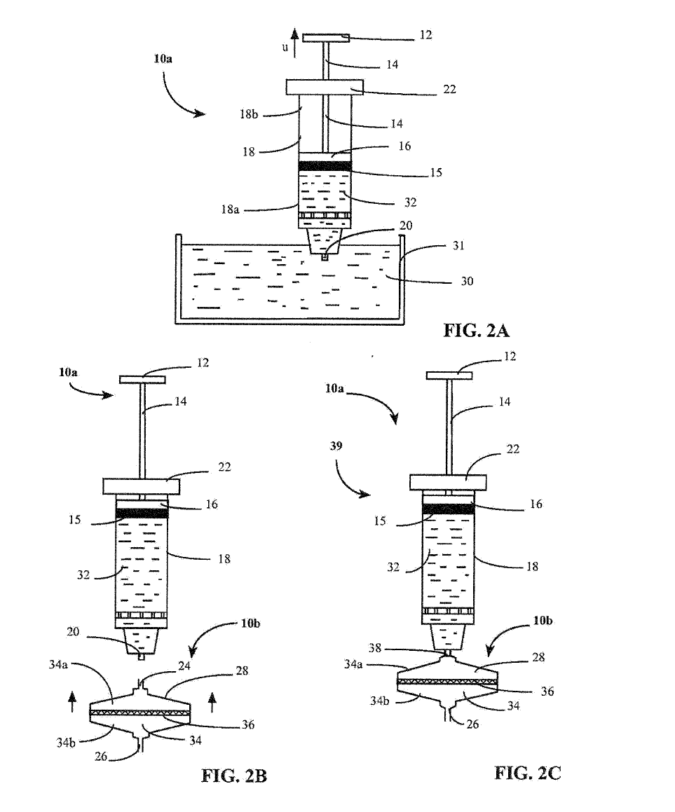

Referring now to FIG. 1, shown therein is a syringe and filter assembly 10, comprising a syringe 10a and a filter unit 10b. The syringe 10a comprises a syringe housing 18, having at its distal end an aperture 20 through which a fluid may enter the syringe 10a or exit from the syringe 10a, the syringe housing 18 further having at its proximal end a flange 22 providing a finger rest and cooperating with the proximal handle comprising finger rest 12. The syringe 10a further comprises a plunger unit 14 longitudinally movable within the syringe housing 18, a piston 16 with a seal 15 providing sealing against the inside wall of the syringe housing 18, and further comprises a proximal handle with a finger rest 12. The diameter of the distal aperture 20 is preferably substantially narrower than the diameter of the syringe housing 18. Thus when downward pressure is applied to the fluid filled syringe housing 18 using the plunger unit 14, a substantial fluid pressure in the narrow distal aperture 20 is achieved. Such pressure is deemed beneficial as it weakens the integrity of the microorganisms' cellular membranes. It is noted that in other embodiments no seal is included. Syringes comprising a seal are also known in the art as BD syringes. Syringes free of a seal are also known in the art as norm-ject syringes. Further shown in FIG. 1 is filter unit 10b having a filter housing 28, with a glass microfiber membrane filter contained therein (not visible), and a filter outlet 26 and a filter inlet 24. The filter inlet 24, in accordance with this embodiment of the disclosure, is constructed to fit to syringe aperture 20 in such a manner that when filter unit 10b and syringe 10a are reversibly coupled via filter inlet 24 and syringe aperture 20, respectively, they together form a reversible joint 38 (see: FIG. 2C) through which fluid communication between fluid present in the syringe housing 18 and the chamber within the filter housing 28 may readily be established. The joint 38 is constructed in such a manner that no fluid leakage occurs during fluid communication between the syringe 10a and filter 10b. Thus in an example embodiment, the reversible joint 38 can be constructed using a screw thread structure.

Referring now to FIG. 2A, the syringe 10a may be used to draw a fluid sample 32 comprising microorganisms from a vessel 31 containing a source fluid 30 into the syringe housing 18 by immersing aperture 20 of the syringe 10a into the fluid 30, while having the piston 16 positioned within the distal portion 18a of the syringe housing 18 and then moving the plunger 14 upward (see: arrow u), thus moving the piston 16 within the syringe housing 18 upward from the distal portion 18a towards the proximal portion 18b of the syringe housing 18, and thereby gradually filling the syringe housing 18 with an aliquot of the source fluid 30 to obtain sample fluid 32.

It is noted that in other embodiments, the syringe 10a can be filled by removing the plunger 14 entirely from the syringe housing 18 and filling the syringe 10a with fluid 30 through the opening at the proximal portion 18b of the syringe 10a. Such an embodiment may be used when the distal portion of the syringe is too wide to access the source fluid 30.

Referring now to FIG. 2B, once a fluid sample 32 of the source fluid 30 has been collected in the syringe 10a, filter unit 10b (shown in cross section) is provided, and reversibly coupled to the syringe 10a via syringe aperture 20 of the syringe 10a and filter inlet 24. Filter unit 10b comprises a filter housing 28 and an interior filter chamber 34 having an upper filter chamber portion 34a separated from a lower filter chamber portion 34b by glass microfiber membrane filter 36 having a pore size sufficiently small to prevent the passage of microorganisms through the membrane filter 36.

Referring now to FIG. 2C, shown therein is filter unit 10b coupled to syringe 10a through joint 38 formed by syringe aperture 20 of the syringe and filter inlet 24, and forming a contiguous assembly 39.

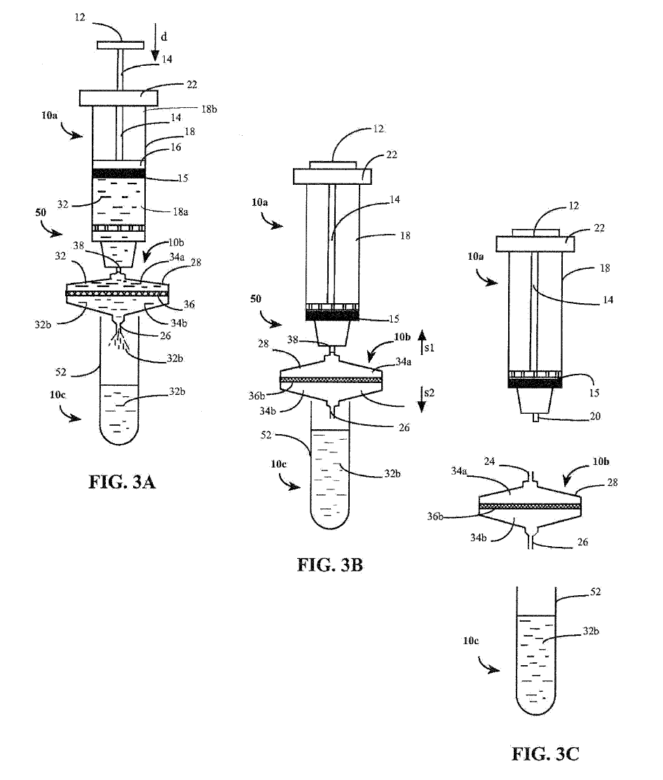

Referring now to FIG. 3A, shown therein is an assembly 50, including syringe 10a, filter unit 10b and a sample collection unit 10c. Sample fluid 32 is filtered by moving plunger 14 downward (see: arrow d), thus moving the piston 16 within the syringe housing 18 downward from the proximal portion 18b towards the distal portion 18a of the syringe housing 18, and thereby gradually emptying the syringe housing 18 and pressing the fluid sample 32 via joint 38 into the upper filter chamber portion 34a, and filtering sample fluid 32b, which upon passage through glass microfiber membrane filter 36 flows through the bottom filter chamber portion 34b and exits the filter unit 10b via filter outlet 26 to be collected in collection vessel 52. Microorganisms are generally unable to pass through filter 36 and hence upon completion of filtering, a glass microfiber membrane filter having microorganisms associated therewith 36b is obtained (as shown in FIG. 3B and FIG. 3C). In some embodiments, the amount of fluid 32b in the collection vessel 52 may be recorded and used for subsequent analysis.

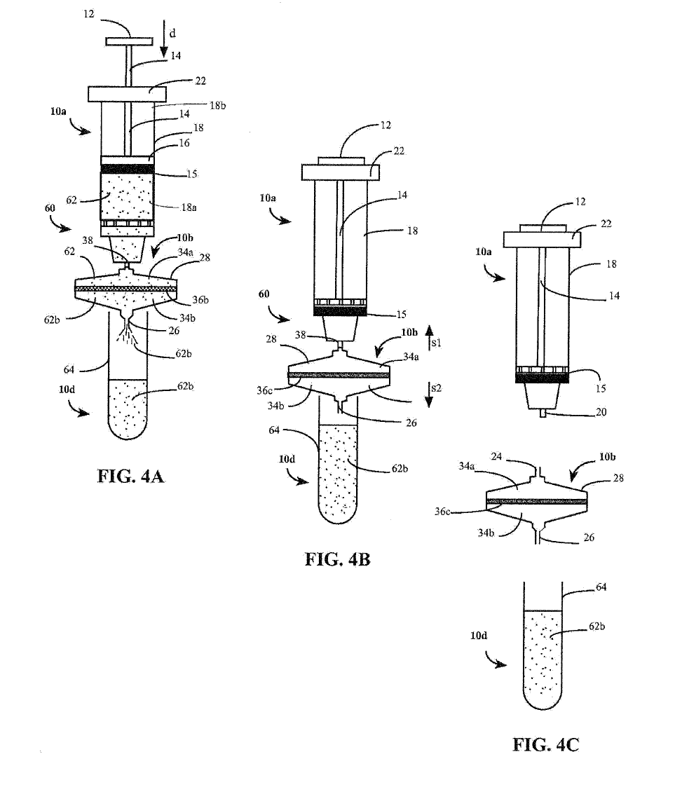

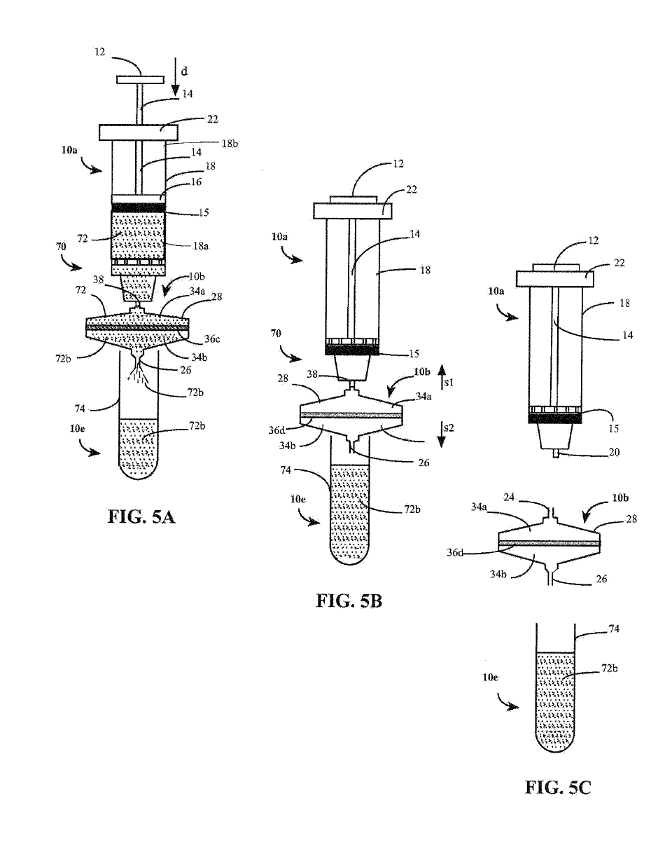

Referring now to FIG. 3B, once piston 16 reaches its most distal position within the syringe housing 18 generally all of the sample fluid 32 will have passed through the filter unit 10b and will be collected in collection vessel 52. Assembly 50, may be disassembled by decoupling the syringe 10a from filter 10b (see: arrow s1), and separating filter unit 10b from the collection vessel 52 (see: arrow s2) containing the filtered fluid sample 32b, in order to obtain a separate syringe 10a, filter unit 10b and collection unit 10c as shown in FIG. 3C. The glass microfiber membrane filter having microorganisms associated therewith 36b may be processed by treating the filter with lysis reagent to lyse the cells, as illustrated in FIG. 4, and thereafter with extractant as further illustrated in FIG. 5.

Referring now to FIG. 4A, shown therein is an assembly 60, containing syringe 10a, filter unit 10b, comprising a glass microfiber membrane filter having microorganisms associated therewith 36b, and a sample collection unit 10d. It is noted that the syringe 10a depicted in FIG. 4 is the same syringe as the syringe depicted in FIG. 3, however it will be clear to those of skill in the art that at this point in the procedure a different syringe may be used. The syringe 10a is filled with a sufficient quantity of lysis reagent 62 which is applied to the filter having microorganisms associated therewith 36b by moving plunger 14 downward (see: arrow d), thus moving the piston 16 within the syringe housing 18 downward from the proximal portion 18b towards the distal portion 18a of the syringe housing 18, and thereby gradually emptying the syringe housing 18 and pressing the lysis reagent 62 via joint 38 into the upper filter chamber portion 34a, through the glass microfiber membrane filter having microorganisms associated therewith 36b, causing the microorganisms to lyse and the nucleic acid material to adhere to the glass microfiber membrane filter 36b. The lysis reagent and non-nucleic acid cellular constituents 62b flow through the bottom filter chamber portion 34b and exit the filter housing 28 via filter outlet 26 to be collected in collection vessel 64. Upon passage of the lysis reagent 62 through the glass microfiber membrane filter 36b, the plunger 14 may be moved repeatedly up and down within the syringe housing 18 to facilitate drying of the glass microfiber membrane filter 36b and removal of substantially all of the lysis reagent 62 from the glass microfiber membrane filter 36b.

Referring now to FIG. 4B, once piston 16 reaches its most distal position within the syringe housing 18, all of the lysis reagent 62 will have passed through the filter unit 10b and will be collected together with non-nucleic acid cellular constituents 62b in collection vessel 64. Assembly 60, may be disassembled by decoupling the syringe 10a from filter 10b (see: arrow s1), and separating filter unit 10b from the collection vessel 64 (see: arrow s2) containing the lysis reagent and non-nucleic acid cellular constituents 62b, in order to obtain a separate syringe 10a, filter unit 10b and collection unit 10d as shown in FIG. 4C. The glass microfiber membrane filter having nucleic acid material adhered thereto 36c may be processed by treating the glass microfiber filter 36c with extractant to as hereinbefore described and further illustrated in FIG. 5A-FIG. 5C.

It is noted that in some embodiments, the filter unit 10b comprising the glass fiber membrane filter with the nucleic acid material adhered thereto 36c, or in other embodiments, the glass fiber membrane filter with the nucleic acid material adhered thereto 36c alone which, depending on the filter unit 10b's design details, may be removed from the housing 28, and is stored for further analysis. It is further noted that the steps illustrated in FIG. 2A to FIG. 4C may be rapidly conducted, requiring, for example, less than 15 minutes to complete. Thereafter filter unit 10b may, if desired, be stored for several hours, or several days, e.g. at least one 1 day, 2 days, 5 days, 10 days or even longer. Thus upon completion of lysis the filter unit 10b may be packaged and transported, for example, from a site at which the steps illustrated in FIG. 2A to FIG. 4C have been performed to another site. The foregoing feature of this embodiment of the present disclosure is particularly beneficial in situations where a fluid sample is obtained at a site or location where laboratory facilities are not readily available, for example, when a water sample is obtained from a body of water located at a remote field site.

It is further noted that in some embodiments, the lysis reagent and non-nucleic acid cellular constituents 62b collected in sample collection vessel 64 may be used for further analysis. In one embodiment, the lysis reagent and non-nucleic acid cellular constituents 62b can be used to evaluate the presence of energy metabolites, such as for example adenosine triphosphate (ATP).

Referring now to FIG. 5A, shown therein is an assembly 70, containing syringe 10a, filter unit 10b, comprising a glass microfiber membrane filter with nucleic acid material adhered thereto 36c, and a sample collection unit 10e. It is noted that the syringe depicted in FIG. 5 is the same syringe as the syringe depicted in FIG. 3 and FIG. 4, however it will be clear to those of skill in the art that at this point in the procedure a different syringe may be used. The syringe 10a is filled with extractant 72 which is applied to the filter with nucleic acid material adhered thereto 36c by moving plunger 14 downward (see: arrow d), thus moving the piston 16 within the syringe housing 18 downward from the proximal portion 18b towards the distal portion 18a of the syringe housing 18, and thereby gradually emptying the syringe housing 18 and pressing the extractant 72 via joint 38 into the upper filter chamber portion 34a, through the glass microfiber membrane filter having nucleic acid material adhered thereto 36c, causing the nucleic acid material to elute from the glass microfiber membrane filter 36c. The resulting nucleic acid material and extractant 72b flows through the bottom filter chamber portion 34b and exits the filter housing 28 via filter outlet 26 to be collected in collection vessel 74.

In an alternate embodiment, extractant may be supplied in an extractant vessel and the syringe 10a having plunger 14 positioned within the distal portion 18a of the filter housing 18 with the filter unit 10b coupled thereto is immersed into extractant. By moving plunger 14 in the proximal direction, extractant enters the filter unit 10b from the extractant vessel through opening 26, then passes through the glass microfiber membrane filter with nucleic acid material adhered thereto 36c, and enters the syringe housing 18 via joint 38, to be collected. The eluate thus collected in the syringe 10a may then be used as hereinbefore described.

Referring now to FIG. 5B, once piston 16 reaches its most distal position within the syringe housing 18, all of the extractant 72 will have passed through the filter unit 10b and the eluate 72b comprising the nucleic acid material will be collected in collection vessel 74. Assembly 70, may be disassembled by decoupling the syringe 10a from filter 10b (see: arrow s1), and separating filter unit 10b from the collection vessel 74 (see: arrow s2) containing the eluate comprising nucleic acid material, in order to obtain a separate syringe 10a, filter unit 10b and collection unit 10e as shown in FIG. 5C. The glass microfiber membrane filter 36d can be substantially free of nucleic acid material. The eluate comprising nucleic acid material 72b is then available for nucleic acid material analysis as hereinbefore described.

In accordance with the foregoing, in another aspect, the present disclosure further provides a method for obtaining nucleic acid material from a source fluid comprising microorganisms, the method comprising:
obtaining in a fluid transfer device a fluid sample from a source fluid comprising microorganisms;
coupling the fluid transfer device to a filter having a pore size sufficiently small to prevent passage of at least some of the microorganisms through the filter;
filtering the fluid sample through the filter thereby collecting the microorganisms on the filter;
decoupling the filter from the fluid transfer device a first time;
coupling a fluid transfer device comprising lysis reagent to the filter;
filtering lysis reagent through the filter to release at least some of the nucleic acid material from the microorganisms on the filter and to adhere the nucleic acid material to the filter; and
decoupling the filter from the fluid transfer device a second time to obtain the nucleic acid material adhered to the filter.

In one embodiment, the fluid transfer device is a syringe.

In one embodiment, the method further comprises an additional step following the step comprising decoupling the filter a second time, the additional step comprising treating the filter with a sufficient quantity of extractant to elute substantially all of the nucleic acid material from the filter and obtain an eluate comprising the nucleic acid material.

In one embodiment, in order to evaluate the quality of a fluid, in addition to evaluating the nucleic acid material present in the extractant, the lysis reagent, once filtered through the filter, is used for evaluation. Hence, in another aspect, in another embodiment the present disclosure further provides a method for evaluating the quality of a fluid, the method comprising:
obtaining in a fluid transfer device a fluid sample obtained from a source fluid comprising microorganisms;
coupling the fluid transfer device to a filter having a pore size sufficiently small to prevent passage of at least some of the microorganisms through the filter;
filtering the fluid sample through the filter thereby collecting the microorganisms on the filter;
decoupling the filter from the fluid transfer device a first time;
coupling a fluid transfer device comprising lysis reagent to the filter;
filtering lysis reagent through the filter to release at least some of the nucleic acid material from the microorganisms on the filter and to adhere the released nucleic acid material to the filter, and to obtain the filtered lysis reagent;
decoupling the filter from the fluid transfer device a second time to obtain a filter having nucleic acid material adhered thereto;
treating the filter with a sufficient quantity of extractant to elute at least some of the nucleic acid material from the filter and to obtain an eluate comprising the at least some of the nucleic acid material; and
analyzing the nucleic acid material in the eluate and analyzing the filtered lysis reagent.

In some embodiments, the filtered lysis reagent is analyzed for the presence of compounds associated with microbial energy such as ATP. Such analysis may, for example, be performed using a luciferase enzyme assay and the spectrophotometric detection of light, based on the following general enzymatic reaction:
luciferin+ATP→luciferyl adenylate+PPi
luciferyl adenylate+O₂→oxyluciferin+AMP+light; and
   as further described by Leach, J. Appl. Biochem., 1981, 3: 473-517, and commercially available through, for example, Promega® (Madison, Wi, USA). It is noted that the foregoing embodiment of the instant disclosure permits the simultaneous gathering of data regarding the nucleic acid constituents of the microorganisms in the source fluid, and the metabolic state of the microorganisms in the source fluid.

In another aspect, the present disclosure provides kits for monitoring water quality and the presence of microorganisms in water. In accordance herewith the kits may be used to obtain nucleic acid material.

Thus in a further embodiment, the present disclosure provides a kit for obtaining a nucleic acid sample, the kit comprising
a fluid transfer device to obtain a fluid sample from a source fluid comprising microorganisms;
a filter having a pore size sufficiently small to prevent passage of at least some of the microorganisms through the filter and to permit collection of the microorganisms associated with the filter; and
a lysis reagent which when filtered through the filter is capable of lysing microorganisms collected on the filter, releasing at least some of the nucleic acid material from the microorganisms, and adhering the released nucleic acid material to the filter.

In some embodiments, the kit further includes an extractant capable of eluting the nucleic acid material from the filter.

The kit, in certain embodiments, can also comprise a washing reagent or instructions for making a washing reagent, in which the combination of the lysis reagent and the extractant allows capture of the microorganisms on the filter for subsequent elution and nucleic acid material analysis. The kit may include more than one type of sampling device, filter, lysis reagent and extractant.

In one embodiment, the kit comprises a syringe to obtain and transfer a fluid sample, and a filter that may reversibly be coupled to the syringe. In one embodiment, the filter included in the kit is a microfiber glass membrane filter.

In another embodiment, the kit further comprises instructional material. As used herein, “instructional material” includes printed matter, e.g., paper or cardboard, a voice or video recording, a diagram, a computer readable medium, such as a disk (floppy diskette or hard disk), optical CD such as CD- or DVD-ROM/RAM, magnetic tape, electrical storage media such as RAM and ROM, and hybrids of these such as magnetic/optical storage media, or any other medium of expression which can be used to communicate the usefulness of the disclosed method(s) or composition(s) for its designated use. For example, the instructional material may comprise instructions on how to collect a fluid sample, how to use the filter, how to make and/or use the lysis reagent, or how to make and/or use the extractant, thus providing guidance to a user of the kit with respect to use the kit's contents. The instructional material of the kit of the present disclosure may, for example, be provided within the kit, affixed to the kit, for example in the form of a label, or attached to a vial or tube containing reagents of the kit, or the instructional material may be separated from the kit but be shipped together with the kit. Alternatively, the instructional material may be shipped separately from the kit, with the intention that the instructional material and the composition be used cooperatively by the recipient.

In further embodiments, the present disclosure provides a use of a kit to evaluate the in situ identity of microorganisms in a fluid, the kit comprising:
a fluid transfer device to obtain a fluid sample from a source fluid comprising microorganisms;
a filter having a pore size sufficiently small to prevent passage of at least some of the microorganisms through the filter and to permit collection of the microorganisms associated with the filter; and
a lysis reagent which when filtered through the filter is capable of lysing microorganisms collected on the filter, release of at least some of the nucleic acid material from the microorganisms, and adherence of the released nucleic acid material to the filter.

The above disclosure generally describes various aspects of processes and uses of the present disclosure. It will be appreciated by a person of skill in the art having carefully considered the above description of representative embodiments of the present disclosure, that a wide variety of modifications, amendments, adjustments, substitution, deletions and other changes may be made to these specific embodiments, without departing from the scope of the present disclosure. Accordingly, the foregoing detailed description is to be understood as given by way of example and illustration only, the spirit and scope of the present disclosure being limited solely by the appended claims.

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

### EXAMPLES

### Example 1—Evaluation of a First Water Sample from an Oil Production Facility

A first water sample was obtained from an oil production facility (sample #1105) and treated and evaluated using the device and method described herein above with respect to FIG. 1-FIG. 5C.

Two volumes of 10 ml each of sample #1105 were drawn into two sterile 60 ml syringes and a sterile filter was coupled to the fluid outlet of each syringe, as described above with respect to FIG. 1-FIG. 5C. Both filters were identical 0.7 μm stacked glass microfiber filters, but processed differently, and referred herein as the “1105-X filter” and “1105-D filter”. The fluid samples were filtered through the respective sterile filters using the syringes. Both filters were then treated with lysis reagent (phosphoric acid, trisodium salt, dodecahydrate, 1-5%; sodium hydroxide, 0.1-1%; alkyl dimethyl benzyl ammonium chloride, 0.1-1%) by passing the lysis reagent through the filters using the syringes. Thereafter a respective syringe volume of air was passed through the filters to remove remaining amounts of lysis reagent and to dry the filters. The filtrates were collected and used for ATP analysis using a luciferase enzyme assay. It is noted that the filtrate was the same 1 ml of lysing agent and the other filtrate was diluted with the 9 ml of Reagent D, and the filters were stored at 0° C. in a self-sealing bag for further treatment and analysis. Following storage, the filters were opened and the DNA was extracted from the filters using a MoBio PowerMag® Soil DNA Isolation kit as the extractant (MoBio Catalog No 2700-4-EP) with a bead beating step.

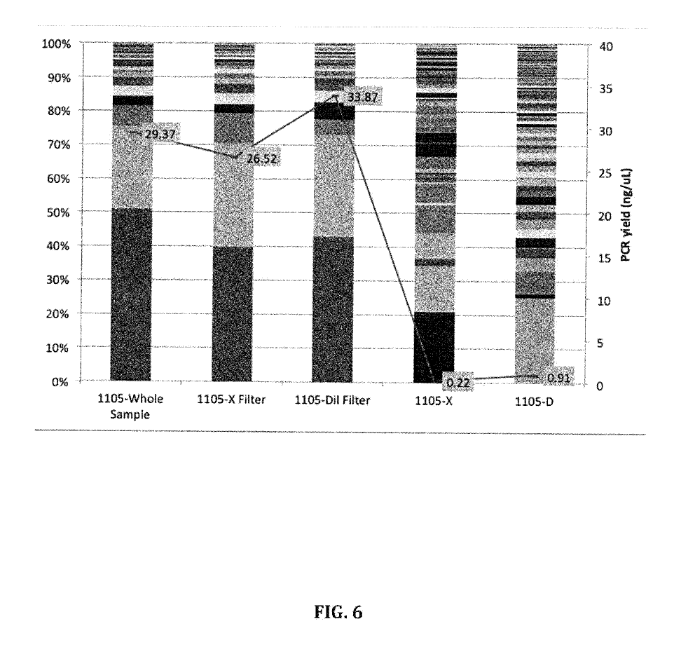

Samples representing various fluid fractions obtained in the performance of the foregoing process (as outlined in Table 1) were subsequently subjected to 16S rRNA sequencing and microbial constituents were evaluated at a genus level. Results of the evaluation are shown in FIG. 6. Referring to FIG. 6, it is noted that the similarity in bar profiles signifies that the constituent microbial genera in the original fluid sample (“1105-Whole Sample”) are similar, both in kind and quantity, to the constituent microbial genera obtained following treatment of the filters with extractant (“1105-X Filter” and “1105-D Filter”). By contrast, the microbial genera present in the filtrate obtained following treatment with lysis reagent (“1105-X”) or a diluted sample thereof (“1105-D”) are very different from those present in the original fluid sample, as signified by the markedly different bar profiles shown in FIG. 6.

Further indicated in FIG. 6 are the PCR yield values. The PCR yield value is a measure of the efficiency and specificity of the PCR reaction and the DNA quality. Higher values represent higher PCR efficiency and DNA concentration, and are indicative of a more accurate sequencing result. In general, for environmental samples, a yield at least 2 ng/μL is considered acceptable. The similar values obtained for samples 1105 Whole Sample, X Filter and Dil Filter indicate that these samples contained similar amounts and quality of DNA, while samples 1105-X and 1105-D did not contain sufficient DNA to obtain accurate sequencing data. Thus the PCR yield values of the 1105-X and 1105-D samples indicate that these samples are less suitable for collecting DNA samples for sequencing.

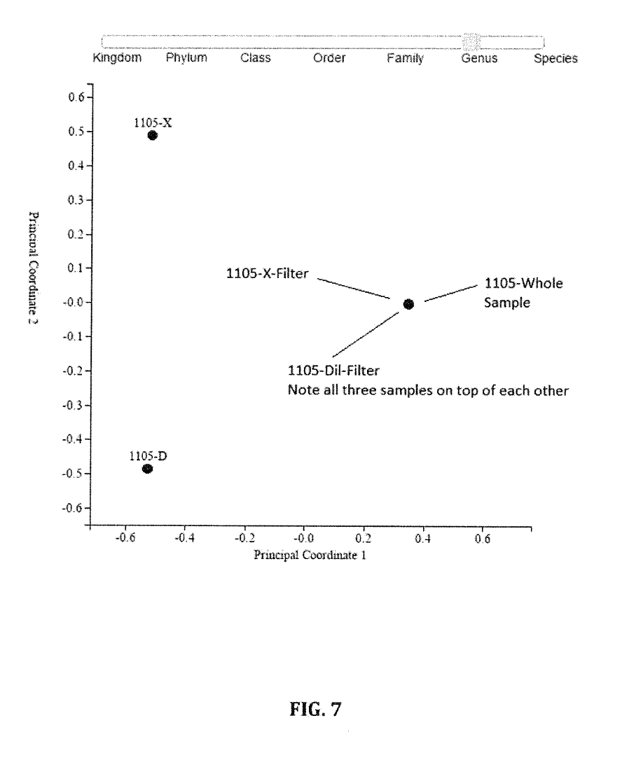

Further results are shown in FIG. 7, which provides a principal component analysis of the various fluid fractions. Principal component analysis is mathematically defined as an orthogonal linear transformation, i.e. a statistical procedure used to explore variation and highlight patterns within complex datasets. This analysis was done using Mothur, an open source program for bioinformatics analysis (Schloss, P. D., et al., Introducing mothur: Open-source, platform-independent, community-supported software for describing and comparing microbial communities. Appl Environ Microbiol, 2009. 75(23):7537-41). The plotting of points relating to the original fluid sample (“1105-Whole Sample”) in close proximity to points relating to the filters (“1105-X filter” and “1105-D filter”) indicates that the microbial compositions of each of these 3 fluid fractions are similar. By contrast, the extract (“1105-X”) and dilution (“1105-D”) plotted at a further distance, indicating a different microbial composition, both relative the original fluid sample and relative to each other.

It is noted that 16s rRNA sequencing identified the presence of the kingdom of Archaea in the original fluid sample (“1105-Whole Sample”), as well as the filters (“1105-X filter” and “1105-D filter”), however Archaea were not identified in the extract (“1105-X”) or dilution (“1105-D”).

Together these results indicate that the filters harbor a microbial composition that is surprisingly reflective of the source fluid sample, however the extract (“1105-X”) and dilution (1105-D”) do not represent the composition of the fluid sample.

The filtrate of the 1105-D sample was used to determine the ATP levels present therein using a luciferase enzyme assay. The results of the assay are shown in Table 2.

TABLE 1
Various fluid fractions used for microbial profile comparisons.

| Sample ID | Description |
|---|---|
| 1105-Whole | DNA sample extracted from the original fluid sample |
| Sample | |
| 1105-X-Filter | Filtrate 1105-X filter following treatment with extractant |
| 1105-D-Filter | Filtrate 1105-D filter following treatment with extractant |
| 1105-X | Filtrate from 1105-X following treatment with lysis |
| | reagent |
| 1105-D | Dilution of filtrate of 1105-X in ultrapure water |
| | following treatment with lysis reagent. |

TABLE 2
ATP analysis.

| Sample ID | Volume ml | Picograms (pg) of ATP/ml |
|---|---|---|
| 1105-D | 10.0 | 173.9 |

### Example 2—Evaluation of a Second Water Sample from an Oil Production Facility

A second water sample was obtained from an oil production facility (sample #1604) and treated and evaluated using the device and method described herein above with respect to FIG. 1-FIG. 5C.

It was determined that the total solids and hydrocarbon in sample #1604 exceeded 10% and/or 100,000 mg/L, respectively. It was therefore deemed desirable to wash the filter prior to treating the filter with lysis reagent. Using two sterile syringes, two respective fluid samples of approximately 20 ml were filtered through two respective 0.7 μm sterile filters having a diameter of approximately 3 cm (“1604-A filter” and “1604-B filter”). The first filtrate samples were collected for analysis. Using the syringes, both filters were then washed using 60-100% dimethyl sulfoxide as a washing reagent. The second filtrates were also collected for analysis. Both filters were then treated with lysis reagent (phosphoric acid, trisodium salt, dodecahydrate, 1-5%; sodium hydroxide, 0.1-1%; alkyl dimethyl benzyl ammonium chloride, 0.1-1%) by passing the lysis reagent through the filters using the respective syringes. Thereafter a syringe volume of air was passed through the filters to remove remaining amounts of lysis reagent and to dry the filters. The final and third filtrates were collected and used for ATP analysis using a luciferase enzyme assay, and the filters were stored at 0° C. in a self-sealing bag for further treatment and analysis. Following storage, the filters were opened and DNA extracted for 16S analysis directly from the surface of the filter using the same procedure as described in Example 1.

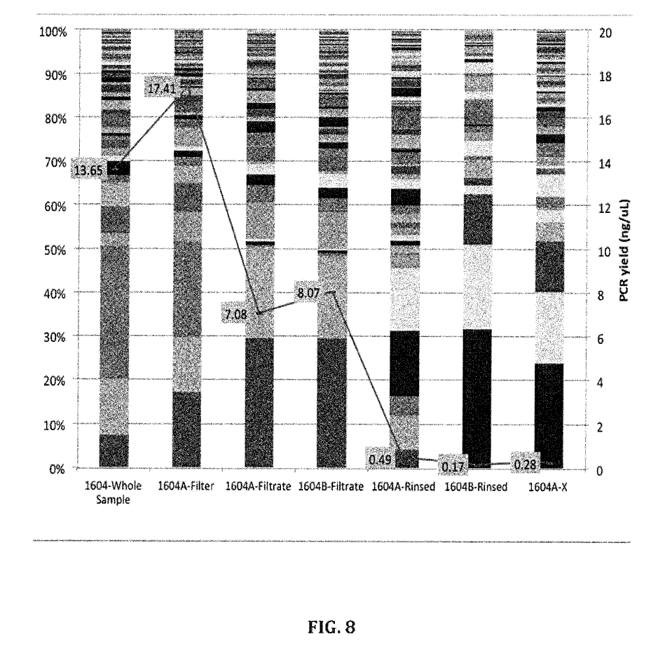

Samples representing various fluid fractions obtained in the performance of the foregoing process (as outlined in Table 3) were subsequently subjected to 16s rRNA sequencing and microbial constituents were evaluated at a genus level. Results of the evaluation are shown in FIG. 8. It is noted that the similarity in bar profiles signifies that the constituent microbial genera in the original fluid sample (“1604-Whole Sample”) is similar, both in kind and quantity, to the constituent microbial genera obtained following treatment of filter A with extractant (“1604A-Filter”). By contrast, the microbial genera present in the other filtrates following are very different from those present in the original fluid sample, as signified by the markedly different bar profiles.

Further indicated in FIG. 8 are the PCR yield values. The PCR yield values of the samples 1604A-Rinsed, 1604B-Rinsed and 1604A-X indicate that these samples are less suitable for collecting DNA samples for sequencing.

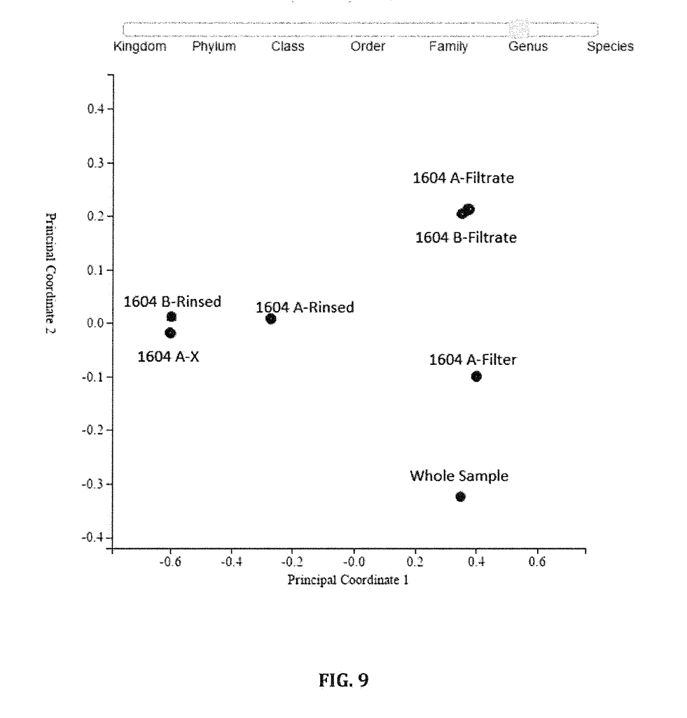

Further results are shown in FIG. 9, which provides a principal component analysis of the various fluid fractions, conducted using Mothur as explained in Example 1. The plotting of the point relating to the original fluid sample (“1604 Whole sample”) in close proximity to the points relating to filter-A (“1604-A filter”) indicates that the microbial profiles between these 2 fluid fractions are similar. By contrast, the other filtrates plotted at a further distance, indicating a different microbial composition, both relative the original fluid sample and relative to each other.

Together these results indicate that the filters harbor a microbial composition that is surprisingly reflective of the fluid sample.

The filtrate of the 1604A-X sample was used to determine the ATP levels present therein using a luciferase enzyme assay. The results of the assay are shown in Table 4.

TABLE 3
Various fluid fractions used for microbial profile comparisons.

| Sample ID | Description |
|---|---|
| 1604-Whole | DNA sample extracted from the original fluid sample |
| sample | |
| 1604A-Filter | Filtrate of 1604-A filter following treatment with |
| | extractant |
| 1604A-Filtrate | First filtrate 1604-A following first filter step |
| 1604B-Filtrate | First filtrate 1604-B following first filter step |
| 1604A-rinsed | Second filtrate from from 1604-A following DMSO wash |
| 1604B-rinsed | Second filtrate from from 1604-B following DMSO wash |
| 1604A-X | Third filtrate of 1604-A following treatment with lysis |
| | reagent. |

TABLE 4
ATP analysis.

| Sample ID | Volume ml | Picograms (pg) of ATP/ml |
|---|---|---|
| 1604A-X | 20.0 | 5.0 |

### Example 3—Evaluation of Water Samples from Multiple Sources

Aqueous samples from multiple industrial site sources were obtained as detailed in Table 5.

TABLE 5
Sample sources.

| Source ID | Description of Sample Source |
|---|---|
| A | Source water |
| D | Hydraulic fracturing flowback water #1 |
| F | Hydraulic fracturing flowback water #2 |
| H | Pond water |
| J | Produced fluid |
| L | Wellhead liquid |
| N | Group inlet separator (post treatment) |
| O | Wastewater injection well |
| X | Oil sands process water |

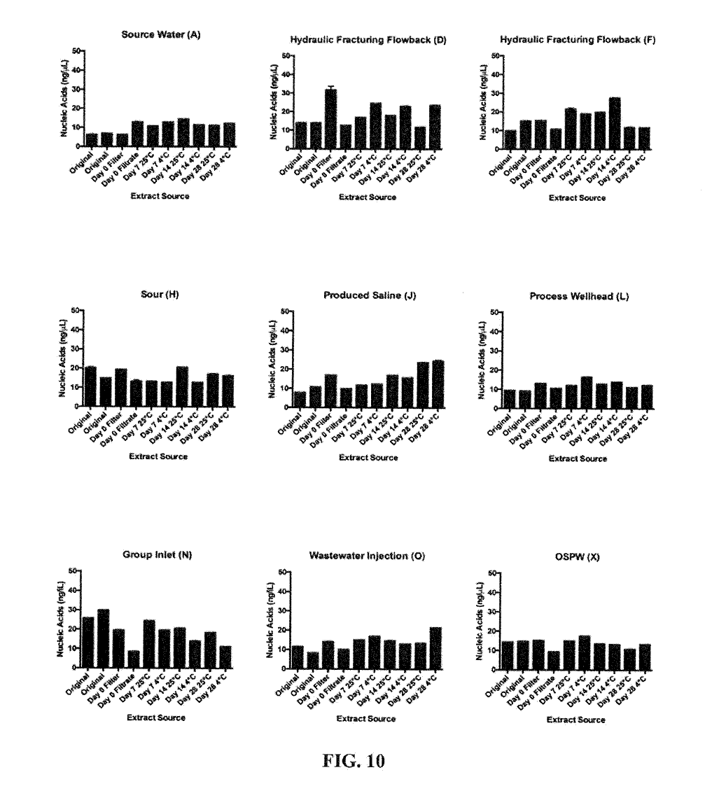

A total of 2×50 ml of samples from each source provided in Table 5 were obtained and bacterial cells were prepared by centrifugation of the sample at 4,000 rpm for 10 minutes and resuspending the cell pellet in 2×200 μl of a 0.9% saline solution to obtain two cell suspensions. One of the two cell suspensions was used to directly quantify nucleic acids present therein. The other cell suspension was filtered, treated with lysis reagent, and thereafter filter-associated nucleic acid material was extracted. Prior to extraction, filters were stored at either 4° C. or 25° C. Nucleic acid extractions were performed either immediately upon filtration or following storage of the filters for a period of 7, 14 or 28 days, at the noted temperatures. Lysis reagent, filters, and extraction reagent were constituted and employed as described in Example 1. Nucleic acid material from the original cell suspension, nucleic acid material extracted from the stored filters, and nucleic acid material from the day-0 filtrate sample was obtained and the nucleic acid concentration in each was determined using a Nanodrop 2000 (Thermo Scientific) (see: FIG. 10). The samples were also subjected to 16S rRNA gene metagenomics sequencing on an Illumina MiSeq platform using universal 16S primers 926f and 1392r (Golby, S et al., 2014, Microbial Ecology 68: 70-80; Ramos-Pádron, 2011, S. et al., Environmental Science & Technol., 45: 439-446; and An D., et al., 2013, Environmental Science & Technol. 47: 10708-10717). Sequencing reads were quality filtered and microorganisms were identified using the GreenGenes database through the Illumina Basespace 16S Metagenomics app. Ver. 1.01 (Illumina Inc.), which is itself an implementation of the Ribosomal Database Project Classifier (Wang, Q. et al., 2007, Applied and Environmental Microbiology, 73: 5261-5267.

Results of measurements to determine the concentration of DNA in various samples are shown in FIG. 10. It is noted that substantial quantities of DNA are present in both the original cell suspension and in the day-0 filtrate sample, indicating that the day-0 filtrate sample is suitable to use for ATP analysis.

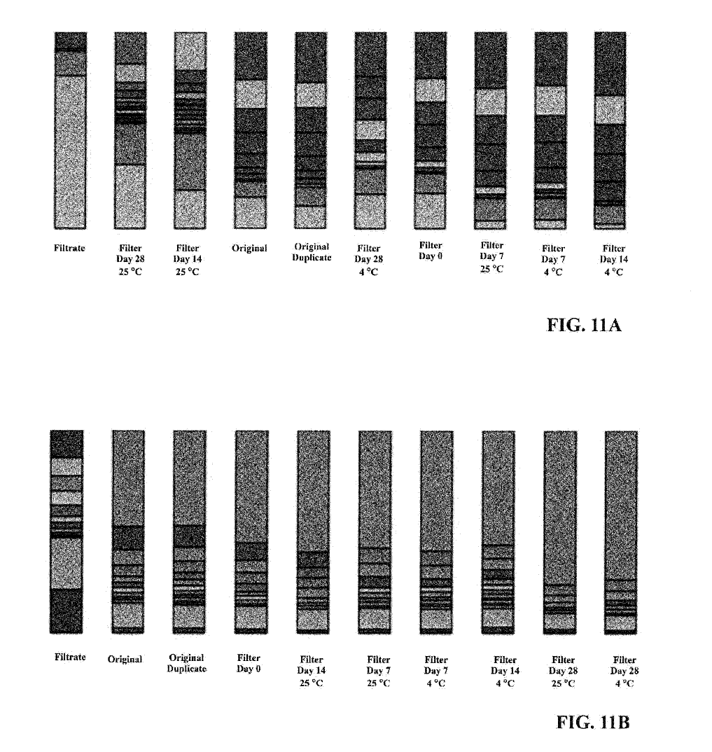

Further results are shown in FIG. 11A-FIG. 11B, showing bar profiles obtained from nucleic acid material from Source ID F and H samples, respectively. As noted in Example 1 similarity in bar profiles signifies that the constituent microbial genera in samples are similar, both in kind and quantity. It is noted that day-0 filtrate profiles in both Source ID F nucleic acid material and Source ID H nucleic acid material differs markedly from bar profiles obtained from any of the filter nucleic acid material. Thus the nucleic acid material identified in the filtrate is not representative for the nucleic acid material present in the original source sample. Furthermore, bar profiles obtained using nucleic acid material from non-stored filters are very similar to bar profiles obtained from the source sample. Thus filter-associated nucleic acid material obtained immediately following sampling is similar to nucleic acid material in the sample. Furthermore bar profiles of nucleic acid material obtained from filters stored at 4° C. remain relatively similar to bar profiles of nucleic material obtained from filters from which nucleic acid material is immediately extracted following sampling. This is in particular the case when filters are stored for 7 or 14 days. However bar profiles of nucleic acid material obtained from filters stored at 25° C., particularly when stored for 14 and 28 days, deviate more significantly from bar profiles of nucleic acid material from filters from which nucleic acid material is immediately extracted following sampling.

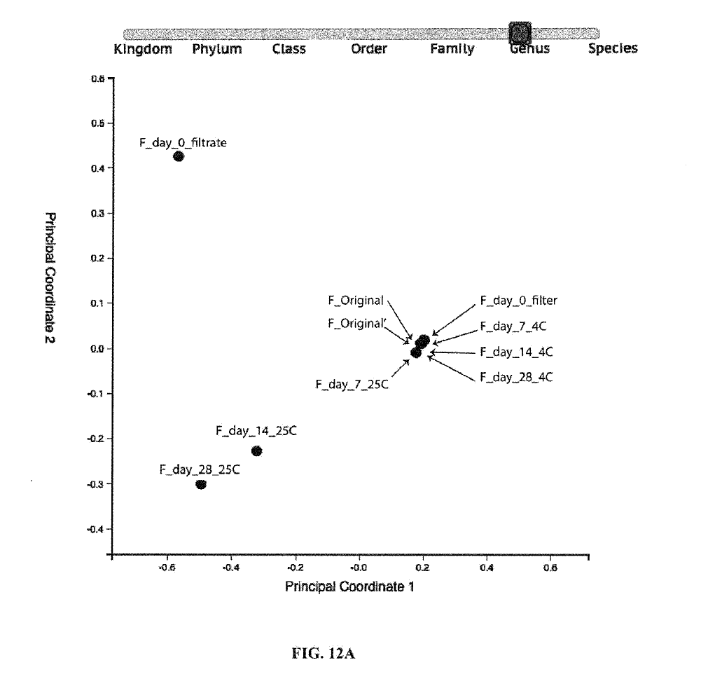

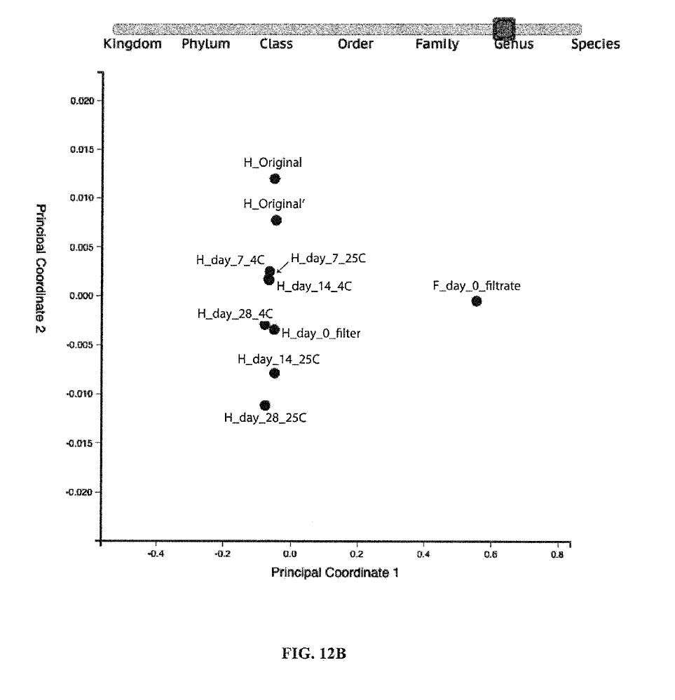

Further results are shown in FIG. 12A-FIG. 12B, which provide a principal component analysis of the various fluid fractions, conducted using Mothur. The plotting of points close to one another indicates that the microbial profiles between two samples are similar. The results shown in the Mothur plots closely correspond with the findings shown in the bar profiles in FIG. 11A-FIG. 11B samples having Source ID F and H.

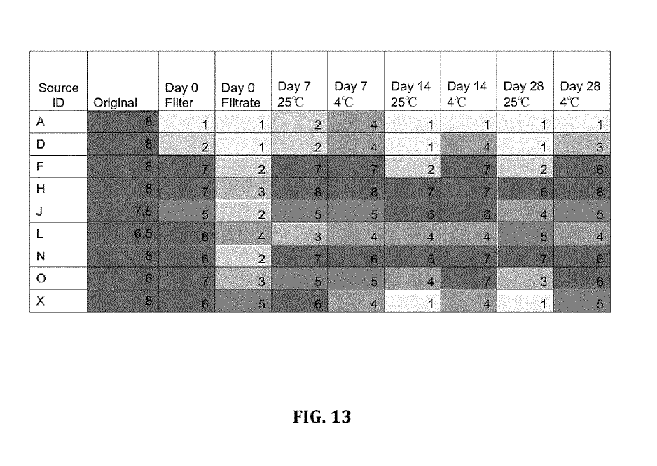

The collective 16S metagenomics data from all 9 sample sources was used to build a heat map as shown in FIG. 13, visually depicting the microbial community similarity of samples at the genus level. For each sample source (original) the top 8 genus classifications were established to serve as the basis from which the top 8 genus classifications for each filter/filtrate sample were compared and scored. In this analysis, instances in which a minimum of 5 of 8 genus classifications matched were coded in a dark grey shade (see: e.g. cell: A/Original) (indicating a high degree of similarity), while less similar communities (<5 matches) were progressively coded towards lighter grey shades (3-4 of 8 genus classifications matched are coded medium dark grey (see e.g. cell: J/Day 0 Filter), two shades of medium light grey (see: e.g. cell H/Day 0 Filtrate; and J/Day 0 filtrate), <3 matches is coded in light grey shade (see: e.g. cell: X/Day 14 25° C.). Several inferences can be made from the results shown in FIG. 13. Filtrate-derived communities are poor representations of the original sample community. Moreover, filter communities, specifically those stored at 4° C. are on the whole adequately representative of original communities (indicated by prevalence of dark shades of grey), although the extent of similarity is source-dependent. Storage of filters at 25° C. allows for microbial community profile shifts, as indicated by the prevalence of light shades of grey (1 match) and medium shades of grey (3-4 matches) for 14 and 28-day storage at this temperature.

Together these results indicate that it is possible to perform the lysis and filtering step on the water sampling site, and that filters subsequently may be stored, for example for up to 14 days before being processed.

### SUMMARY OF SEQUENCES

SEQ ID NO: 1 is a synthetic polynucleotide primer known as 8F
SEQ ID NO: 2 is a synthetic polynucleotide primer known as U1492R
SEQ ID NO: 3 is a synthetic polynucleotide primer known as 928F
SEQ ID NO: 4 is a synthetic polynucleotide primer known as 336R
SEQ ID NO: 5 is a synthetic polynucleotide primer known as 1100F
SEQ ID NO: 6 is a synthetic polynucleotide primer known as 1100R
SEQ ID NO: 7 is a synthetic polynucleotide primer known as 337F
SEQ ID NO: 8 is a synthetic polynucleotide primer known as 907R
SEQ ID NO: 9 is a synthetic polynucleotide primer known as 785F
SEQ ID NO: 10 is a synthetic polynucleotide primer known as 805R
SEQ ID NO: 11 is a synthetic polynucleotide primer known as 533F
SEQ ID NO: 12 is a synthetic polynucleotide primer known as 518R
SEQ ID NO: 13 is a synthetic polynucleotide primer known as 27F
SEQ ID NO: 14 is a synthetic polynucleotide primer known as 1429R

1. A method for obtaining nucleic acid material from a source fluid comprising microorganisms, the method comprising:
obtaining a fluid sample from a source fluid comprising microorganisms;
filtering the fluid sample through a filter having a pore size sufficiently small to prevent passage of at least some of the microorganisms through the filter to collect microorganisms associated with the filter;
treating the filter with a sufficient quantity of lysis reagent to release at least some of the nucleic acid material from the microorganisms associated with the filter and for the released nucleic acid material to adhere to the filter; and
treating the filter with a sufficient quantity of extractant to elute at least some of the nucleic acid material from the filter and to obtain an eluate comprising the nucleic acid material from the filter.

2. The method according to claim 1 wherein the source fluid is a natural or man-made body of water.

3. The method according to claim 1 or claim 2 wherein the filter is a glass microfiber filter.

4. The method according to any one of claims 1 to 3 wherein the lysis reagent includes at least one of Triton X-100; Triton X-114; NP-40; Brij-35; Brij-58; Tween 20; Tween 80; octyl glucoside; octyl thioglucoside; sodium dodecyl sulfate (SDS); 3-[(3-cholamidopropyl)dimethylammonio]-l-propanesulfonate (CHAPS); 3-[(3-cholamidopropyl)dimethylammonio]-2-hydroxy-l-propanesulfonate (CHAPSO); alkyldimethylbenzylammonium chloride (ADBAC); didecyldimethylbenzylammonium chloride (DDAC); peptidoglycan lyase; β-N-acetylmuramidase; and β-l, 3-glucanase; tris(2-carboxyethyl)phosphine (TCEP) and dithiothreitol (DTT).

5. The method according to any one of claims 1 to 4 wherein the extractant is Tris-EDTA (TE) or distilled water.

6. The method according to any one of claims 1 to 5 wherein the filtering occurs under pressure.

7. A method for evaluating the in situ quality of a fluid, the method comprising:
obtaining a fluid sample from a source fluid comprising microorganisms;
filtering the fluid sample through a filter having a pore size sufficiently small to prevent passage of at least some of the microorganisms through the filter to collect microorganisms associated with the filter;
treating the filter with a sufficient quantity of lysis reagent to release nucleic acid material from at least some of the microorganisms associated with the filter and for the released nucleic acid material to adhere to the filter;
treating the filter with a sufficient quantity of extractant to elute at least some of the nucleic acid material from the filter and to obtain an eluate comprising the at least some of the nucleic acid material; and
analyzing the nucleic acid material in the eluate.

8. The method according to claim 7 wherein the fluid sample is obtained from a body of water and the method is a method for evaluating the in situ quality of the body of water.

9. The method according to claim 7 or claim 8 wherein the filter is a glass microfiber filter.

10. The method according to any one of claims 7 to 9 wherein the lysis reagent includes at least one of Triton X-100; Triton X-114; NP-40; Brij-35; Brij-58; Tween 20; Tween 80; octyl glucoside; octyl thioglucoside; sodium dodecyl sulfate (SDS); 3-[(3-cholamidopropyl)dimethylammonio]-l-propanesulfonate (CHAPS); 3-[(3-cholamidopropyl)dimethylammonio]-2-hydroxy-l-propanesulfonate (CHAPSO); alkyldimethylbenzylammonium chloride (ADBAC); didecyldimethylbenzylammonium chloride (DDAC); peptidoglycan lyase; β-N-acetylmuramidase; and β-l, 3-glucanase; tris(2-carboxyethyl)phosphine (TCEP) and dithiothreitol (DTT).

11. The method according to any one of claims 7 to 10 wherein the extractant is Tris-EDTA (TE) or distilled water.

12. The method according to any one of claims 7 to 11, wherein analyzing the nucleic acid material in the eluate comprises identification of a class to which microorganisms present in the eluate belong.

13. The method according to claim 12 wherein analyzing the nucleic acid material in the eluate comprises amplifying the nucleic acid material by PCR.

14. The method according to claim 12 wherein analyzing the nucleic acid material in the eluate comprises obtaining nucleic acid sequence data.

15. The method according to claim 12 wherein analyzing the nucleic acid material in the eluate comprises obtaining 16S rRNA nucleic acid sequence data.

16. The method according to claim 12 wherein analyzing the nucleic acid material in the eluate comprises obtaining 16S rRNA nucleic acid sequence data, and comparing the obtained sequence data with 16S rRNA nucleic acid sequence data present in a database comprising known 16S rRNA sequences.

17. The method according to any one of claims 7 to 16 further involving a step of identifying and recording one or more classes of microorganisms present in situ in the source fluid.

18. The method according to claim 17 wherein the classes of microorganisms are recorded and stored on a computer readable medium.

19. The method according to any one of claims 14 to 16 wherein the data are recorded and stored on a computer readable medium.

20. The method according to any one of claims 7 to 19 wherein the filtering occurs under pressure.

21. A method for obtaining nucleic acid material from a source fluid comprising microorganisms, the method comprising:
obtaining in a fluid transfer device a fluid sample from a source fluid comprising microorganisms;
coupling the fluid transfer device to a filter having a pore size sufficiently small to prevent passage of at least some of the microorganisms through the filter;
filtering the fluid sample through the filter thereby collecting the microorganisms on the filter;
decoupling the filter from the fluid transfer device a first time;
coupling a fluid transfer device comprising lysis reagent to the filter;
filtering lysis reagent through the filter to release at least some of the nucleic acid material from the microorganisms on the filter and to adhere the nucleic acid material to the filter; and
decoupling the filter from the fluid transfer device a second time to obtain the nucleic acid material adhered to the filter.

22. The method according to claim 21 wherein the fluid transfer device is a syringe.

23. The method according to claim 21 or claim 22 further comprising treating the filter comprising nucleic acid material with a sufficient quantity of extractant to elute at least some of the nucleic acid material from the filter and to obtain an eluate comprising the at least some of the nucleic acid material.

24. The method according to any one of claims 21 to 23 wherein
upon filtering the lysis reagent to obtain the filtered lysis reagent, the method comprises:
treating the filter comprising nucleic acid material with a sufficient quantity of extractant to elute at least some of the nucleic acid material from the filter and to obtain an eluate comprising the at least some of the nucleic acid material; and
analyzing the nucleic acid material in the eluate and the filtered lysis reagent.

25. The method according to 24 wherein the method comprises analyzing the filtered lysis reagent for the presence of ATP.

26. The method according to any one of claims 21 to 25 wherein the source fluid is an aqueous fluid.

27. The method according to any one of claims 21 to 25 wherein the source fluid is a natural or man-made body of water.

28. The method according to any one of claims 21 to 27 wherein the filter is a glass microfiber filter.

29. The method according to any one of claims 21 to 28 wherein the lysis reagent includes at least one of Triton X-100; Triton X-114; NP-40; Brij-35; Brij-58; Tween 20; Tween 80; octyl glucoside; octyl thioglucoside; sodium dodecyl sulfate (SDS); 3-[(3-cholamidopropyl)dimethylammonio]-l-propanesulfonate (CHAPS); 3-[(3-cholamidopropyl)dimethylammonio]-2-hydroxy-l-propanesulfonate (CHAPSO); alkyldimethylbenzylammonium chloride (ADBAC); didecyldimethylbenzylammonium chloride (DDAC); peptidoglycan lyase; β-N-acetylmuramidase; and β-l, 3-glucanase; tris(2-carboxyethyl)phosphine (TCEP) and dithiothreitol (DTT).

30. The method according to any one of claims 21 to 25 wherein the extractant is Tris-EDTA (TE) or distilled water.

31. A kit for obtaining a nucleic acid sample, the kit comprising
a fluid transfer device to obtain a fluid sample from a source fluid comprising microorganisms;
a filter having a pore size sufficiently small to prevent passage of the microorganisms through the filter and to permit collection of the microorganisms associated with the filter; and
a lysis reagent which when filtered through the filter is capable of lysing microorganisms collected on the filter, releasing at least some of the nucleic acid material from the microorganisms, and adhering the nucleic acid material to the filter.

32. The kit according to claim 31 wherein the kit further comprises an extractant for eluting of the nucleic acid material to obtain an eluate comprising nucleic acid material.

33. The kit according to claim 31 or claim 32 wherein the fluid transfer device is a syringe and wherein the filter is reversibly coupled to the syringe.

34. The kit according to any one of claims 32 to 33, the kit further comprising instructional material.

35. The kit according to claim 34 wherein the instructional material comprises instructions describing (i) how to collect the fluid sample, (ii) how to use the filter, or (iii) how to make and/or use the lysis reagent.

36. The kit according to any one of claims 31 to 35 wherein the filter is a glass microfiber filter.

37. The kit according to any one of claims 31 to 36 wherein the lysis reagent includes at least one of Triton X-100; Triton X-114; NP-40; Brij-35; Brij-58; Tween 20; Tween 80; octyl glucoside; octyl thioglucoside; sodium dodecyl sulfate (SDS); 3-[(3-cholamidopropyl)dimethylammonio]-l-propanesulfonate (CHAPS); 3-[(3-cholamidopropyl)dimethylammonio]-2-hydroxy-l-propanesulfonate (CHAPSO); alkyldimethylbenzylammonium chloride (ADBAC); didecyldimethylbenzylammonium chloride (DDAC); peptidoglycan lyase; β-N-acetylmuramidase; and β-l, 3-glucanase; tris(2-carboxyethyl)phosphine (TCEP) and dithiothreitol (DTT).

38. The kit according to claim 32 wherein the extractant is Tris-EDTA (TE) or distilled water.

39. A use of a kit to evaluate the in situ identity of microorganisms in a fluid, the kit comprising:
a fluid transfer device to obtain a fluid sample from a source fluid comprising microorganisms;
a filter having a pore size sufficiently small to prevent passage of at least some of the microorganisms through the filter and to permit collection of the microorganisms associated with the filter; and
a lysis reagent which when filtered through the filter is capable of lysing the microorganisms collected on the filter, release of at least some nucleic acid material from the microorganisms, and adherence of the released nucleic acid material to the filter.

40. A use of a kit according to claim 39 wherein the kit further comprises an extractant for elution of the nucleic acid material to obtain an eluate comprising nucleic acid material.

41. A use of a kit according to claim 39 or claim 40 wherein the fluid transfer device is a syringe and wherein the filter may reversibly be coupled to the syringe.

42. A use of a kit according to any one of claims 39 to 41, the kit further comprising instructional material.

43. A use of a kit according to claim 42 wherein the instructional material comprises instructions describing (i) how to collect the fluid sample, (ii) how to use the filter, or (iii) how to make and/or use the lysis reagent.

44. A use of a kit according to any one of claims 39 to 43 wherein the filter is a glass microfiber filter.

45. A use of a kit according to any one of claims 39 to 44 wherein the lysis reagent includes at least one of Triton X-100; Triton X-114; NP-40; Brij-35; Brij-58; Tween 20; Tween 80; octyl glucoside; octyl thioglucoside; sodium dodecyl sulfate (SDS); 3-[(3-cholamidopropyl)dimethylammonio]-l-propanesulfonate (CHAPS); 3-[(3-cholamidopropyl)dimethylammonio]-2-hydroxy-l-propanesulfonate (CHAPSO); alkyldimethylbenzylammonium chloride (ADBAC); didecyldimethylbenzylammonium chloride (DDAC); peptidoglycan lyase; β-N-acetylmuramidase; and β-l, 3-glucanase; tris(2-carboxyethyl)phosphine (TCEP) and dithiothreitol (DTT).
